(19) United States
(12) Patent Application Publication
(10) Pub. No.: US 2026/0051412 A1
(43) Pub. Date: Feb. 19, 2026

(54) CLINICIAN RESPONSIBLE AI COMPANION

(71) Applicant: Movius Interactive Corporation, Alpharetta, GA (US)

(72) Inventors: Amit Modi, Fremont, GA (US); Swetha Manjappa, Bengaluru (IN); ARAVINDKUMAR ABBENA, Bengaluru (IN)

(73) Assignee: Movius Interactive Corporation, Alpharetta, GA (US)

(21) Appl. No.: 18/809,176

(22) Filed: Aug. 19, 2024

(51) Int. Cl. G16H 80/00; G06F 40/166; G06F 40/295; G06F 40/35; G06F 40/58; H04L 51/046; H04L 51/216; H04L 51/52

(52) U.S. Cl. CPC G16H 80/00; G06F 40/166; G06F 40/295; G06F 40/35; G06F 40/58; H04L 51/046; H04L 51/216; H04L 51/52

(57) ABSTRACT

A distributed system for collaboration of the operations of a single or multiple healthcare provider entities, and more particularly, a distributed healthcare system and method that operates to provide unification of communications, siloed and fragmented data harmonization, remoteness and decentralization exacerbating the need for a unique solution to address new age healthcare needs, authentication for security and privacy and real-time data sharing.

### CROSS-SECTION TO RELATED APPLICATIONS

This application incorporates and implements certain technology found in U.S. Pat. No. 9,332,128 issued on May 3, 2016, U.S. Pat. No. 10,542,395 issued on Jan. 21, 2020, U.S. Pat. No. 11,032,427 issued on Jun. 8, 2021, and U.S. Pat. No. 11,563,711 issued on Jan. 24, 2023, as well as U.S. application Ser. No. 18/391,390 filed on Dec. 20, 2023 and having the title of “DECENTRALIZED BLOCKCHAIN ENABLED MOBILE COMMUNICATIONS ON A SECURE, OPEN AND DISTRIBUTED NETWORK”, and U.S. patent application Ser. No. 18/809,032 bearing the title of PURPOSE DRIVEN SECURE COMMUNICATIONS PLATFORM and attorney docket number 07001.1930gss, filed concurrently herewith on Aug. 19, 2024. All of the above are hereby incorporated by reference in their entirety.

### BACKGROUND

The geographically dispersed nature of patients, imbalance between demand and local availability of clinicians and care providers, the pervasiveness of manual processes and documentation, and fragmented healthcare ecosystem (lacking the benefits of Artificial Intelligence, data harmonization and real time data sharing) present a tremendous opportunity for advancements.

I am sure you have heard the old proverb “necessity is the mother of invention”. This proverb has been applied to Aesop's famous fable “The Crow and the Pitcher” where a thirsty crow happens up a pitcher that is partially filled with water. But the crow cannot reach down into the pitcher to get a drink. The crow then begins collecting pebbles to drop into the pitcher, thereby causing the water to rise to a level where the thirsty crow can have a drink. But is this proverb always true?

In many instances throughout life, inventions have been made well before their time of usefulness or feasibility. The one I think we are all waiting on is the STAR TREK teleporting system. Other inventions that were given birth to decades before they found practical applications and implementations include the jet pack, contact lenses, solar cells and vending machines. In addition, there are also many examples in which invention became the mother for necessity rather than necessity being the mother of invention. This occurs when a technology emerges to solve one problem, but then creates, one or more, or even a myriad of other problems. T.

### SUMMARY OF THE DISCLOSURE

The various embodiments of the present invention are directed towards a distributed system for collaboration of the operations of a single or multiple healthcare provider entities, and more particularly, a distributed healthcare system and method that operates to provide unification of communications, siloed and fragmented data harmonization, remoteness and decentralization exacerbating the need for a unique solution to address new age healthcare needs, authentication for security and privacy and real-time data sharing.

The various embodiments are enabled, in one aspect, by utilizing MultiLine service developed by MOVIUS INTERACTIVE INC. The MultiLine service is a powerful yet easy to use software application that puts a dedicated HIPAA compliant business number on any smart phone or tablet. As such, the use of MultiLine in the various embodiment provides healthcare professionals (HCPs), sales reps, and clinical trial staff with a secure way to separate their work and personal calls and text/SMS/MMS messages all from the same device. The MultiLine service hosts powerful features like opt in and opt out for text messages, redaction of sensitive information, one-to-one and group messaging, configuring business hours, call scheduling, forwarding and dedicated voice mail. MultiLine service also works with MICROSOFT TEAMS, thus enabling a company's staff to work from anywhere with voice and text capabilities right from their laptop, tablet or smart phone with a seamless integrating within the Microsoft Teams application.

The various embodiments are further enhanced by the fact that the MultiLine service is fully functional with wearable devices, thus providing enhanced patient monitoring and safety protocols. MultiLine service integrates into social messaging platforms like WHATSAPP, WECHAT and LINE, so providers can communicate with patients in more ways from more places.

All voice and SMS communications using the MultiLine service can be captured and stored in the cloud for easy retrieval ensuring that the service providers remain fully HIPAA compliant at all times. The employment of the MultiLine service allows this functionality at a fraction of the price of traditional or fixed wireless services or costly employee phone stipends.

The employment of the MultiLine service enhances telehealth and provider real-time patient engagement. Healthcare provider staff, such as doctors/nurses/clinicians/etc. can enhance the care provided both in the clinical setting and in clinical studies in person and remotely. The healthcare provider loads the MultiLine service app onto their communication device, such as a smart phone, tablet, computer, etc. The patient can continue to use their existing communications devices and channels (such as SMS, WHATSAPP, Voice Calls and Video (with integrations)), which advantageously removes significant barriers to engagement. With the MultiLine service, the providers can thus contact the patients who receive the contact requests on their devices and using applications that they are familiar and comfortable use.

The embodiments are also based on the use of a single unique identification number assigned or associated with communication devices in conjunction with a communications platform that enables cross-communication between various platforms and apps in a seamless and integrated fashion.

The embodiments also incorporate the use of state-of-the-art artificial intelligence (AI) to perform analysis, modeling and predictive actions based on the data received by the system.

In an exemplary embodiment, the health care services are integrated into a distributed system or provisioned in a method. As a method, the services are integrated by providing access to a communications platform over a network. The communications platform comprises an artificial intelligence (“AI”) tool for processing received information and communications. Further, the method includes providing communication devices, such as but not limited to mobile devices, that are serviced or provisioned by or for the communications platform to a plurality of healthcare workers. Each of the mobile devices includes a unique identifier.

In operation, a health service request is received at the communications platform. The health service request is generally sent from an originating mobile device and comprises a unique identifier associated with the originating mobile device. The health service request may be a doctor-patient conversation, a healthcare provider-healthcare provider conversation, a healthcare provider monitoring a wearable device of a patient, and a hospital worker receiving information from a remote service facility, as non-limiting examples.

Based on the health service request, the communication platform establishes a communication channel between the originating mobile device and one or more participating devices. For instance, the originating device may be utilized by a doctor or other healthcare provider and the one or more participating devices by be utilized by a patient, doctor, healthcare worker, etc. It should also be appreciated that a patient may initiate the health service request. The process of setting up the communications channel, in some embodiments involves: identifying a communication protocol for the originating mobile device; identifying a communication protocol for the one or more participating devices; and performing any conversion necessary for the participating devices to understand the communication transmission from the originating device and vice versa.

Subsequent to establishing the communication channel, all communicated information is routed through the communications platform and processed by the AI tool. As should be expected, much of the communicated information is in the form of voice. The various embodiments perform the action of creating a text file by performing a speech to text translation on voice information received in the communication information over the communication channel.

The AI tool operates to analyze the received communicated information to make assessments related to the received communicated information. Some examples of operations that can be performed by the AI too include: (a) performing diarization to identify speakers for portions of the text in the text file; (b) analyzing the received communicated information by creating a summarization; (c) analyzing the received communicated information by performing a sentiment analysis; (d) analyzing the received communicated information by performing emotion detection; (e) analyzing the received communicated information by performing named entity recognition; (f) analyzing the received communicated information by performing topic modeling; and (g) analyzing the received communicated information by performing language translation.

The communication platform also interfaces with an electronic healthcare system to provide the received communicated information and the assessments. As such, the received communication information, along with all of the analysis performed by the AI tool, can be stored in a history file and accessed by other devices. The use of authentication in the devices advantageously satisfies requirements under HIPPA. Further, the system operates by receiving a next health service request at the communications platform from a next originating mobile device. The next health service request also includes a unique identifier associated with the next originating mobile device. Based on the health service request, the communication platform establishes a communication channel between the next originating mobile device and one or more participating devices. When the next health service request is to access previously stored data within the electronic healthcare system, the data is thus shared in real-time with authorized users

In the various embodiments, the one or more participating devices may include one or more voice enabled internet of things (“IOT”) devices and the communications platform may receive communicated information from the voice enabled IOT devices. Further, in various embodiments, the communications platform comprises a second line service. As such, the devices are equipped with a second line for interfacing and interacting with the health care system.

Further embodiments include a system for integrating the provision of health care services. The system includes a communications platform accessible over a network and including an interface to an electronic healthcare system. Further, the system includes an artificial intelligence tool configured to process received information and communications. The system utilizes and communications with a plurality of mobile devices that are provisioned and or serviced by the communications platform. Each such mobile device includes a unique identifier. The communications platform includes an executable software programs stored within memory accessible to the communications platform, which when executed causes the communications platform to receive a health service request from an originating mobile device and including the unique identifier associated with the originating mobile device. Based on the health service request, the communication platform establishes a communication channel between the originating mobile device and one or more participating mobile devices.

Subsequent to establishing the communication channel, all communicated information is sent through the AI tool for being analyzed to make assessments related to the received communicated information. Further, the received communicated information and the assessments are then provided to the interface of the electronic healthcare system. The system further may include a speech to text translator to convert voice information received in the communication information over the communication channel into a text file. Further, the system may also include a diarization system to identify speakers for portions of the text in the text file. In various embodiments, the communications platform comprises a second line service and is further configured to provision the mobile devices with the second line service and receive health service requests through the second line service.

These and other features and embodiments are described in greater detail in conjunction with the figures and the herein after provided detailed description provided.

### BRIEF DESCRIPTION OF THE DRAWINGS

In the Figures, like reference numerals refer to like parts throughout the various views unless otherwise indicated. For reference numerals with letter character designations such as “102A” or “102B”, the letter character designations may differentiate two like parts or elements present in the same figure. Letter character designations for reference numerals may be omitted when it is intended that a reference numeral encompass all parts having the same reference numeral in all figures.

FIG. 1 is a functional diagram illustrating the telecommunications integration goal of various embodiments of the present invention.

FIG. 2 is a functional block diagram illustrating the operation of an exemplary communications platform interacting with a carrier partner providing certain integrated communication services.

FIG. 3 is a flow diagram illustrating a first communication exchange example based on an exemplary architecture such as that illustrated in FIG. 2 as a non-limiting example.

FIG. 4 is a flow diagram illustrating a communication exchange example based on an exemplary architecture such as that illustrated in FIG. 2.

FIG. 5 is a flow diagram illustrating another communication exchange example based on an exemplary architecture such as that illustrated in FIG. 2.

FIG. 6 is a sky of clouds illustrating the overall operation of an embodiment of an exemplary communications platform 602.

FIG. 7 is a functional block diagram of the components of an exemplary embodiment of system or sub-system operating as a controller or processor 700 that could be used in various embodiments of the disclosure for controlling aspects of the various embodiments.

FIG. 8 is a conceptual diagram illustrating the connected provider ecosystem that can be achieved through deployment of embodiments of the present invention.

FIG. 9 is a conceptual diagram illustrating a summary of the solutions provided by the various embodiments of the present invention.

FIG. 10 is a conceptual block diagram illustrating the use of an AI tool within various embodiments of the CRAC.

### DETAILED DESCRIPTION

The various embodiments of the present invention are directed to the provision of a decentralized healthcare solution, and more specifically, to providing a secure communications, clinical data sharing, and AI powered real-time or near real-time intelligence to enhance the quality and personalization of patient care at significantly lower cost. The power of secure, reliable, accessible, and remotely available real-time communication and clinical data sharing provided by the various embodiments advantageously: (1) enhances the patient's care experience; (2) improves the provider's care delivery efficiency and experience; and (3) enhances the cost effectiveness of care.

The approach presented in one or more of the various embodiments focuses on several industry problems and provide solutions to problems such as: (1) Provider Patient Real Time Engagement; (2) Remote Patient Monitoring; (3) Clinician Workflow Automation; (4) Clinical Data Sharing; and (5) Work Personal Life Compartmentalization and Privacy.

Advantageously, the various embodiments of the present invention provide such solutions and more, in an ethical, auditable, scalable and compliant manner.

The various embodiments of the present invention are collectively referred to herein as a Clinician Responsible AI Companion or CRAC. It will be appreciated that the features, functions, aspects, operations, capabilities, advantages, etc. that are presented herein may be included or excluded by the various embodiments. As such, each embodiment described, unless stated otherwise, may include or exclude one or more of the described features, functions, aspects, operations, capabilities, advantages, etc. without regard to which specific embodiment they were described.

The various embodiments of the CRAC generally leverage several technologies to provide the system and service. These technologies include: (1) the Single Line Service (SLS) or Multi-Line Service (MLS) available from MOVIUS INTERACTIVE CORPORATION as described in U.S. Pat. No. 9,332,128 (incorporated herein-above by reference); combined with (2) the Single Unique Identity technology described in U.S. Patent Application______having the title of PURPOSE DRIVEN SECURE COMMUNICATIONS PLATFORM incorporated herein-above by reference; along with (3) artificial intelligence and clinical data sharing such as available with the CLINIX AI and CLARE products developed by MOVIUS INTERACTIVE CORPORATION and as described in U.S. application Ser. No. 18/391,390 incorporated herein-above by reference.

Some of the features that may be incorporated into the various embodiments include, but are not limited to: transcription, translation (multi-lingual), summarization, medically made relevant, speaker identification, speaker diarization, AI based actions trigger for clinicians, clinical data sharing, report patient monitoring and quality of life automation. Advantageously, the various embodiments address healthcare needs by providing (a) human centered personalization of care, at scale; (b) AI powered efficiency and automation to reduce cognitive and physical overhead; (c) secure and HIPAA compliant communications; (d) ecosystem integration with E H R/E M R systems and other communication and collaboration tools; and (c) connectivity fabric: cellular/private/wi-fi network.

Navigating secure and reliable communications in the healthcare and pharmaceutical space can be costly and complex. The embodiments of the present invention focus on enhancing the quality of care and improving patient to healthcare provider engagement, while making it both easy and cost efficient for employers. Advantageously, the various embodiments enhance the quality of patient care, improve health care provider efficiency, automate clinician workflow, and reduce per capita cost per care and connect more people from more places.

### MultiLine Service

The various embodiments are enabled, in one aspect, by utilizing MultiLine service developed by MOVIUS INTERACTIVE INC. The MultiLine service is a powerful yet easy to use software application that puts a dedicated HIPAA compliant business number on any smart phone or tablet. As such, the use of MultiLine in the various embodiment provides healthcare professionals (HCPs), sales reps, and clinical trial staff with a secure way to separate their work and personal calls and text/SMS/MMS messages all from the same device. The MultiLine service hosts powerful features like opt in and opt out for text messages, redaction of sensitive information, one-to-one and group messaging, configuring business hours, call scheduling, forwarding and dedicated voice mail. MultiLine service also works with MICROSOFT TEAMS, thus enabling a company's staff to work from anywhere with voice and text capabilities right from their laptop, tablet or smart phone with a seamless integrating within the Microsoft Teams application.

The various embodiments are further enhanced by the fact that the MultiLine service is fully functional with wearable devices, thus providing enhanced patient monitoring and safety protocols. MultiLine service integrates into social messaging platforms like WHATSAPP, WECHAT and LINE, so providers can communicate with patients in more ways from more places.

All voice and SMS communications using the MultiLine service can be captured and stored in the cloud for easy retrieval ensuring that the service providers remain fully HIPAA compliant at all times. The employment of the MultiLine service allows this functionality at a fraction of the price of traditional or fixed wireless services or costly employee phone stipends.

The employment of the MultiLine service enhances telehealth and provider real-time patient engagement. Healthcare provider staff, such as doctors/nurses/clinicians/etc. can enhance the care provided both in the clinical setting and in clinical studies in person and remotely. The healthcare provider loads the MultiLine service app onto their communication device, such as a smart phone, tablet, computer, etc. The patient can continue to use their existing communications devices and channels (such as SMS, WHATSAPP, Voice Calls and Video (with integrations)), which advantageously removes significant barriers to engagement. With the MultiLine service, the providers can thus contact the patients who receive the contact requests on their devices and using applications that they are familiar and comfortable use.

### CLARE AI Tool

Trial clinicians can take advantage of powerful AI tools to enhance the experience and functionality. Various AI tools can be used to automate documenting and analyzing conversations involving providers and/or patients including capturing relevant case history, scientifically categorizing the discussion, patient sentiment and emotional analysis, and can provide a raw transcript of conversations (including local languages) for review. These provider/patient conversations can be revisited for review and input from multiple physicians at different locations.

One such AI tool is CLARE, developed by MOVIUS INTERACTIVE INC, which is a robust call sentiment analyzing tool that when combined with MultiLine, opens up new possibilities for remote patient monitoring and remote clinical trials by processing patient results data at scale. Having these tools already functional and available enables companies to keep their medication or device launch on track and on budget.

The various embodiments of the CRAC provide remote patient monitoring. The CRAC system is able to securely transmit data collected remotely via wearable and nonwearable Internet of Things (IOT) devices in near real-time. The ability to transmit health information in near real-time advantageously enables the triggering of emergency communication and alerts. These intelligent edge devices are also capable of detecting ambient noise like a screech, a scream, a specific call for help, and other untowardly incidents (falls, absence of movement, change in sound patterns, etc.) that may require emergent attention.

In addition, the various embodiments of the CRAC enable clinician workflow automation with efficient documentation and focus on the patient. One of the big challenges for medical practitioners is the enormity of time spent in note taking (“the pajama shift”). Utilizing CRAC, the doctor-patient, as well as doctor-doctor face-to-face interactions can be captured in real-time and case history can be provided, thereby improving turn-around time for doctors and enabling them to see more patients, while making interactions and capturing case history more accurately and effectively. This also drives a much higher quality of record keeping including historical notes, historical data, current notes and the ability of being able to refer to those easily. The CLARE AI powered software application securely and privately listens, records, transcribes, and translates the conversation. It then also applies AI to create medically relevant summaries thus automating the entire workflow. The data generated from Natural Language Processing and summaries can then be shared across systems using Application Programming Interfaces (APIs) and securely over a decentralized network. These summaries can constitute a comprehensive medical history including (but are not limited to) patient demographic information, symptoms and problem list, preexisting conditions, concomitant medications, habits.

The various embodiments of the CRAC also include the ability to perform clinical data sharing and collaboration. Using the MultiLine service, laboratory reports, Digital Imaging and Communications in Medicine (DICOM) images, and pathology and procedure reports and images, etc., can be transmitted in near real-time enabling physicians to make clinical decisions more efficiently and effectively and enhancing patient access to high quality care. Those skilled in the art will appreciate that transmission of such information is highly regulated by HIPAA requirements and thus it is imperative that the information is transmitted and stored in a secure manner. One technique for providing this security is through the use of blockchain technology, such as the technology described in U.S. patent application Ser. No. 18/391,390 herein incorporated above by reference. This technology provides security through a decentralized and distributed communications fabric that is Blockchain Enabled. This ground-breaking innovation can securely bring together health care records, patient status in real-time, the locally available practitioner and medical help over a connected “chain” without compromising privacy and authenticity. Furthermore, in healthcare audit, this blockchain serves as an immutable set of records (ledgers). While today, when claims come through medicolegal cases credibility and accountability is missing. This new paradigm demonstrates traceability, accountability and auditability and can be enabled across channels (i.e. WHATSAPP, Voice Calls, Text messages and Video engagements, etc.).

The various embodiments of the CRAC also provide compartmentalization of the work versus private life for HCP workers and practitioners, as well as privacy. For instance, the use of MultiLine allows doctors/providers to utilize their own devices that are equipped with an independent and secure phone number for work-related communications. Providers no longer sacrifice their privacy when communicating with patients or others via their mobile devices. Moreover, the entire conversation between a doctor and a patient or other doctor can be recorded in a secured platform with AI capturing case history; adding to accountability and compliance.

In summary, Clinical Data Sharing can not only be encouraged but be practically advanced in the real world by combining the powers of these five components:
1) Non-blockable, geographically dispersed and available secure communications infrastructure;
2) Connected and intelligent AI powered mobile endpoints (Medical devices, mobile phones, tablets, wearables, IoT devices, and more) with ambient condition detection and communication capability;
3) An efficient and automated AI function enabling automated written translation and transcription of oral communication;
4) A flexible platform with rich APIs and data layer to integrate with Electronic Medical (Health) Records for AI powered automation; and
5) A mobile application that compartmentalizes professional and personal communication independently of one another and enables omni-channel communication

Advantageously, this results in a higher quality of care and a lower cost to patients while enhancing the efficiency of the care providers and providing improved frictionless experience to patients.

Turning now to the figures in which like elements are represented by similar labels, various embodiments, as well as aspects, features and characteristics of the embodiments are presented in more detail.

The heart of today's telecommunication industry is found in wireless based networks serving as the integration of mobile and wireless devices into the historical public switched network technology (“PSTN”). However, the use of the PSTN to reach into homes and businesses has been greatly replaced using wireless technology, while the backbone of the PSTN is still in use. But wireless and mobile networks are quite varied and exist in a variety of telecommunications technologies, such as 3G, 4G, 5G, LTE, GSM, etc., as well as other wireless technology including WIFI, BLUETOOTH, INFRARED, BROADCAST RADIO, MICROWAVE, and even satellite technologies. Each of these technologies have similarities and differences.

A goal or object of the various embodiments of the present invention is to provide a technique, technology, solution, methodology, etc. that enables the widest array of end user devices to communicate with other end user devices and provide compatibility no matter what protocol or technology being utilized. Throughout this description, the term end user device is used to refer to any element that can originate and/or receive a communication. Thus, as non-limiting examples, end user devices (sometimes referred to herein as EUDs) may be mobile telephones, smart phones, tablets, note pads, IPADS, notebook computers, desktop computers, personal data assistants, POTS devices, applications running on any of the afore mentioned devices such as messaging applications, video conference applications, voice messaging applications, financial transaction devices, point of sale terminals, CRM applications, social media applications, PBXs, archival applications, enterprise collaboration applications, healthcare integrations, etc. As such, a EUD may be an application running on a mobile telephone, or it may be a hardware enablement of a mobile telephone, etc.

### Single Unique Identity

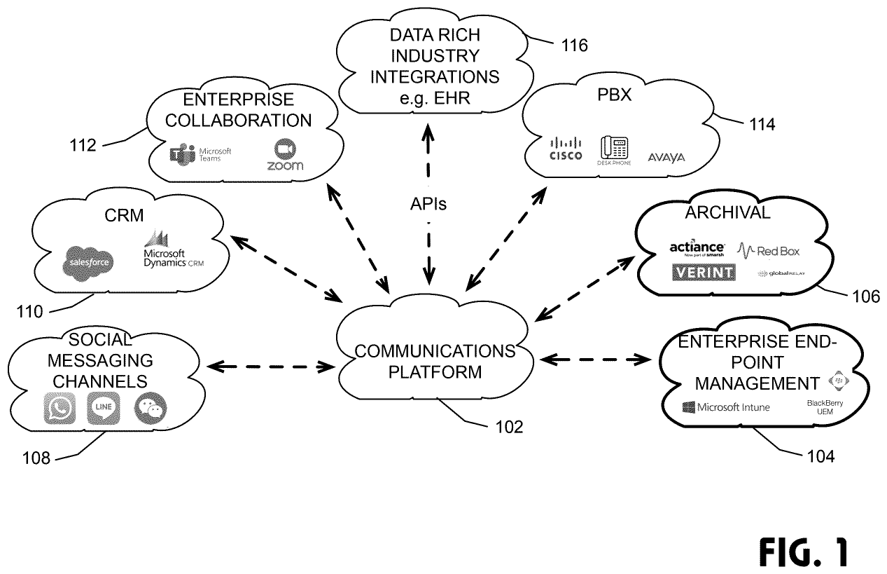

FIG. 1 is a functional diagram illustrating the telecommunications integration goal of various embodiments of the present invention. The listing of various communication sources, sinks, participants, etc., should not be viewed as comprehensive but rather as illustrative of the versatility of the various embodiments.

At the core of the integrated telecommunications illustrated in FIG. 1 (communications platform 102) is the traditional voice and SMS (short message service) or MMS (Multimedia Messaging Service). However, in addition to those two fundamental channels of communication (reference 102), the disruption of the various embodiments present invention may also bring together, from left to right:
(1) Multiple social messaging channels 108, such as WhatsApp, WeChat, Line (as non-limiting examples) all tied to this single unique number and without needing separate application interfaces OR separate identifiers;
(2) CRM cloud 110 illustrates the extension of the ability to receive or send calls and messages (include SMS, MMS, Social Messaging) within a multitude of CRM (Customer Relationship Management) systems without needing separate phone numbers or identities;
(3) Enterprise collaboration cloud 112 illustrates the extension of the ability for the users to be able to use the same phone number or identity within Unified Communication solutions (like MS Teams or Zoom or Cisco Webex or others) such that users can now spend their time productively without switching interfaces to connect with different people in their preferred ecosystems;
(4) PBX cloud 114 illustrates the seamless integration with PBX (Private Branch Exchange) systems eliminating the need for multiple phone numbers and multiple islands of communication (Fixed vs. Mobile); and
(5) Last but certainly not the least, security is preserved at each endpoint, at each interface and at each interaction with seamless integrations into Enterprise Mobility Management Solutions—104.

All of these above-described interactions can then be seamlessly integrated and ingested contextually and tied to that single unique identifier into the applicable System of Records systems like: Electronic Health Records (E H R) systems in health care 116; and a multitude of archival platforms used in several commercial and defense systems 106.

If we take a step back, and envision this disruption, this single number (identity) traversing multiple disparate communication islands and technologies would become the holy grail of simplification (as opposed to increasing the complexity), unification (as opposed to fragmentation), data amalgamation (as opposed to data silos), accessibility of information (as opposed to context switching from technology to technology) and bringing the notion of the identity tied to a position in a business not a specific individual, and that identity can be transferred seamlessly (people change, positions remain). All of this is achieved without creating a telecommunication standard or network specific solution.

A communications platform 102 is illustrated as being the central agent for enabling cross-communication among various sources, sinks, participants and technologies. The communications platform 102 may be an artificial intelligence (AI) powered, purpose driven, secure communications platform. The communications platform 102 can operate in a personal domain, an enterprise or business domain, or a hybrid of the two. Within the personal domain, the communications platform 102 seamlessly integrates the communications activity of a user including activities such as social media posts, social messaging channels, calendar activities, household management activities such as notifications from connected houses (i.e., ALEXA, SIRI, GO GOOGLE, SAMSUNG SMART THINGS, etc.), family schedules, school notifications, etc.

Within the business domain, the communications platform 102 seamlessly integrates into enterprise workflows and SaaS apps.

A workflow is a system for managing repetitive processes and tasks which occur in a particular order. They are the mechanism by which people and enterprises accomplish their work, whether manufacturing a product, providing a service, processing information or any other value-generating activity.

Within business process management, a workflow can be defined as a simple series of individual tasks, while a business process is considered more complex, consisting of multiple workflows, information systems, data, people and their activity patterns. A workflow is distinguished by its simplicity and repeatability, and it is generally visualized with diagram or checklist.

Workflow management software assists in simplifying and optimizing a business process within an organization. It largely does this by coordinating interactions among different stakeholders or between individuals and information systems. Workflow management systems route tasks to the appropriate employee at the right time, providing the pertinent information and nudge to expedite work along the overall process. It also supports manual and automated tasks through document management for activities, like expense reports.

Saas (Software As A Service) is a form of cloud computing in which the provider offers the use of application software to a client and manages all the physical and software resources used by the application. The distinguishing feature of SaaS compared to other software delivery models is that it separates the possession and ownership of software from its use.

As such, the communications platform 102 operates to tie in a wide variety of business activities that operate in the realm of workflows and SaaS. For instance, one such activity includes enterprise end-point management 104. Examples of this activity include Microsoft Intune, which is a cloud-based unified endpoint management service for both corporate and BYOD devices. It extends some of the “on-premises” functionality of Microsoft Endpoint Configuration Manager to the Microsoft Azure cloud. Another example includes the BlackBerry UEM, which delivers complete, unified endpoint management and policy control for a company's diverse and growing fleet of devices and apps. The BlackBerry UEM includes a single management console and trusted end-to-end security model. The BlackBerry UEM is designed to help increase the productivity of a company's mobile workforce while ensuring the full protection of the company's business data.

The communications platform 102 also facilitates archive integration 106. The communications platform 102 enables cloud-based recording of communications and data. The communications platform 102 enables the cloud-based recording to accessed by any EUD and thus, provides compliance, gap-free recording of messages regardless of the location of the EUD. This capability also allows the EUD to transfer recorded communications and data to preferred archival solutions for long-term retention and to have a level of confidence in the securement, retention and accuracy of recorded communications and messaging data. Advantageously, this capability simplifies the supervision, analysis, reporting and eDiscovery of the communications and data. Recordings, files, message streams, etc. can be automatically uploaded to any archival solution from any EUD utilizing the communications platform. Archival solutions may include DUBBER, VERINT, GLOBAL RELAY, VERBA, NICE, SOTERIA, REDBOX, ACTIANCE, AND SPLUNK, as non-limiting examples.

The communications platform 102 also facilitates the consolidation of business mobile messaging channels 108. Messages can be exchanged with consumers on multiple platforms, texting and social messaging, including WhatsApp, WeChat, and LINE etc. in a seamless and integrated manner. Advantageously, EUD can thus communicate with any customer over any of the messaging channels without requiring the customer to adapt to the messaging channel of the EUD and also giving the EUD the freedom to utilize a preferred messaging channel. Further, this aspect of the communications platform 102, regardless of if the EUD is an enterprise device or a BYOD, provides more ways in which clients can keep in touch, maintains professionalism by enabling messaging using official business accounts, and avoids the hassle, expense and time waste of utilizing multiple EUDs.

It should be appreciated that the communications platform 102 also facilitates a cross-blend or combination of the various integrations. For instance, consolidating mobile messaging channels 108 also is integrated with the recordation of messages and the archiving of the same 106. Message recording includes messages, picture messaging, group messaging, and automated messages, such as opt-ins. For instance, an opt-in asks for and captures consent send messages from your business. Opt-in and texting disclaimers can be generated and delivered according to local requirements. In addition, redaction, the action of automatically preventing prohibited terms or information from being shared over messages or stored in your archives can also be integrated across the various channels and EUDs.

Along the same lines, many companies still employ the utilization of PBX systems for inter and extra company communications. This is especially true for work forces that do not have to be on the go but rather are able to be positioned at their stations, desks or offices. The communications platform 102 facilitates the consolidation of business messaging and communication channels through the use of PBX systems 114. Thus, state of the art PBX systems such as CISCO and AVAYA, as non-limiting examples, are seamlessly integrated by means of the communications platform 102.

Advantageously, the communications platform 102 provides seamless mobility in the personal and business domains. In the business domain, users can send messages from their desktop phones, desktop computers, MICROSOFT TEAMS, SALESFORCE CRM, etc. Thus, the employees can message at their desk or on the go with mobile EUDs. Employees can efficiently send messages utilizing their computer keyboard by using Desktop or Microsoft Teams integrations.

It can thus be appreciated that the communications platform 102 also greatly facilitates seamlessly integrating CRM activities 110. CRM is typically used to refer to customer relationship management software that provides the ability to track each interaction that employees have with prospects or customers. This interaction can include activities such as sales calls, customer service interactions, marketing emails, strategic brainstorming among colleagues, and more. CRM tools can unify customer and company data from many sources and even use AI (artificial intelligence) to help better manage relationships across the entire customer lifecycle, spanning departments like marketing, sales, digital commerce, and customer service interactions. The communications platform 102 thus seamless integrates a variety of commercially available CRM systems such as SALESFORCE, MICROSOFT DYNAMICS 365, HUBSPOT, PIPEDRIVE, MONDAY SALES CRM, etc. Thus, regardless of the EUD utilized for CRM, embodiments of the communications platform 102 may enable the data to be accessible to any other EUD.

An ever-increasing focus in today's workplace stems around the phrase “enterprise collaboration”. Enterprise collaboration is the process of helping diverse employees engage in teamwork across borders such that remote and local workers can participate in day-to-day tasks like file sharing, project management, and social media, all through one cohesive online system. With the influx of technology companies trying to create and market the “perfect enterprise collaboration” system, companies have acquired and adopted several solutions such as MICROSOFT TEAMS, SKYPE, ZOOM, etc. Another advantage of the communications platform 102 is the ability to seamlessly integrate various enterprise collaboration systems 112.

In addition to all the above, there are many additional industries that are classified as “data rich” industries, meaning that their day-to-day operations generate, collect, store, and depend on large amounts of data. Typically, this data needs to be reliably stored, securely protected and easily accessible by authorized EUDs. Advantageously, the embodiments of the communications platform 102 seamlessly integrate data rich industries 116, such as Electronic Health Records (EHR) in the health industry, banking and securities, media and entertainment, pharma and healthcare, education, manufacturing, insurance, transportation, government, energy and utilities, and retail and wholesale as non-limiting examples.

It should be understood by anyone in the industry, mobile messaging is a reality of doing business and it is going to be around for a long time. As such, it is imperative that companies give their employees a way to do business, easily and compliantly. The embodiments of the present invention provide technical solutions and platforms in which one or more of the afore-described advantages and aspects can be achieved. The various embodiments of the communications platform provide the afore-described seamless integration through the use of a single number. In general, in one embodiment, each EUD that is serviced by the communications platform 102 is associated with a single unique number. That number is used in connection with all forms of communication with that EUD and the unique numbers can be tracked on behalf of various entities. For instance, on a personal level, a user may utilize 10 EUDs for various communication needs. The unique numbers associated with these EUDs all result in causing the telecommunications infrastructure to forward control of all such communications for these EUDs to the communications platform 102. At the communications platform 102, all these 10 unique numbers associated with the users 10 EUDs can be all tied together as being related to the same user. As such, the communications platform 102 can seamlessly integrate all communications associated with these EUDs.

In other embodiments, the EUDs owned or utilized by a particular entity may all include an identification number associated with that entity. As such, all communications that utilize that unique identification number can be seamlessly integrated by the communications platform 102.

In yet other embodiments, each of the EUDs may include unique identification numbers and the communications platform 102 can include various rules and categorizations to associate various unique identification numbers with various entities. For instance, Table 1 illustrates how a communications platform 102 could associate various unique identification numbers with various entities.

TABLE 1

| | | | | Associated |
|---|---|---|---|---|
| | | Associated | Associated | with User A |
| Unique ID | | with User A | with User B | and B's |
| Number | EUD | personal | personal | employer |
| UIDN0001 | User A's | Yes | No | Yes |
| | BYOD phone | | | |
| UIDN0002 | User B's | No | Yes | Yes |
| | BYOD phone | | | |
| UIDN0003 | User A's | Yes | No | Yes |
| | ZOOM account | | | |
| UIDN0004 | User A's | Yes | No | No |
| | FACEBOOK | | | |
| | acct | | | |

In this simplified example, the communications platform 102 associates User A's BYOD phone, User B's BYOD phone and User A's ZOOM account with the employer of User A and User B. In addition, User A's BYOD phone, User A's ZOOM account and User A's FACEBOOK account are all associated with User A personally. Likewise, User B's BYOD phone is personally associated with User B.

As such, in some embodiments, all EUDs associated with a user or an entity may utilize the same unique ID for that user or entity or, each EUD may have a unique ID and the communications platform 102 can associate one or more of the unique IDs with various users and/or entities. In the former case, communications associated with EUDs and identified by the unique IDs may also include further information to identify the identity of the type of EUD so that the communications platform can process the data appropriately (i.e., translate a FACEBOOK messenger such that it can be received by a WHATSAPP app).

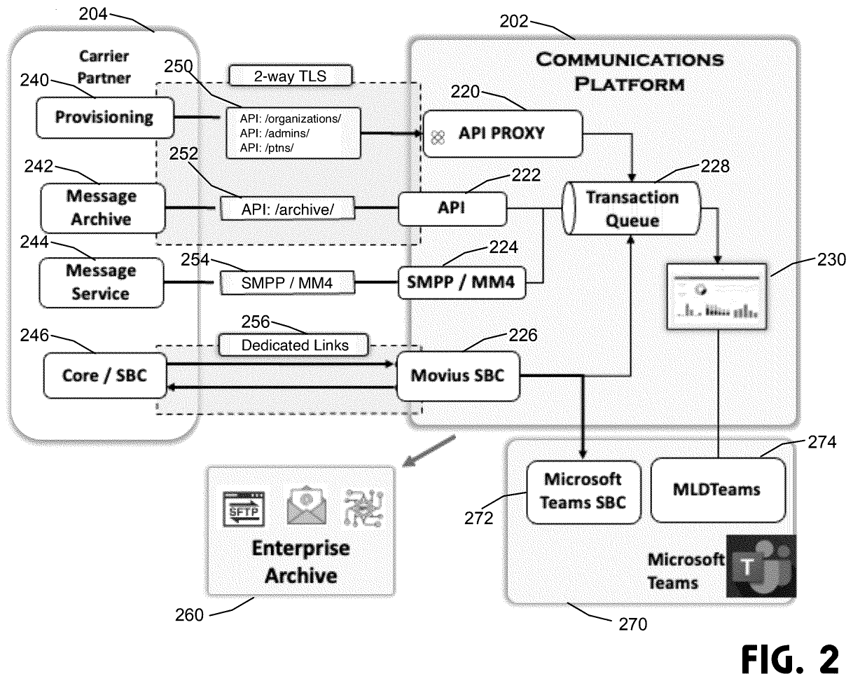

FIG. 2 is a functional block diagram illustrating the operation of an exemplary communications platform interacting with a carrier partner providing certain integrated communication services. The exemplary communications platform 202 is illustrated as interfacing with a carrier partner 204. It should be appreciated that the carrier partner 204 can be any participating carrier, such as brand name mobile provides (i.e., T-MOBILE) as well as small tier equipment lessees that also provide wireless service or any other communications or technology company that may be equipped to receive communication requests initiated by EUDs. Further, it should be understood, that while one carrier partner is illustrated in FIG. 2 multiple carrier partners may exist simultaneously and interface to the communications platform 202.

Initially the Carrier Partner 204 provisions the EUDs that are slated to receive or that are subscribed to the communications integration services provided through various embodiments of the present invention. As such, the Carrier Partner 204 operates initially to provision customers, admins, and lines. The Carrier Partner 204 provides the provisioning of the customers, admis, and lines by employment of a provisioning function 240 by the Carrier Partner 204 what interfaces with an API (Application Protocol Interface) proxy 220 provided by the communications platform 202 over one or more 2-way TLS communication channels.

TLS or Transport layer security is a cryptographic protocol that provides end-to-end security of data sent between applications over the Internet. TLS evolved from secure socket layers (SSL), which NETSCAPE COMMUNICATIONS CORPORATION developed in 1994 to secure web sessions.

TLS is normally implemented on top of transmission control protocol (TCP) in order to encrypt application layer protocols such as HTTP, file transfer protocol (FTP), simple mail transfer protocol (SMTP) and internet message application protocol (IMAP), although it can also be implemented on user datagram protocol (UDP), datagram congestion control protocol (DCCP) and stream control transmission protocol (SCTP), as well.

The Carrier Partner 204 operates to provision for the integrated communications service provided through the communications platform 202 for any form of EUD that is leveraging the service. In essence, during the provisioning process the Carrier Partner 204 receives a unique identifier that is associated with the EUD. The Carrier Partner 204 then associates that unique identifier with that particular EUD and tags the EUD and unique identifier as being services by the communications platform 202. As a result of the provisioning, any and all forms of communication that are directed through the Carrier Partner 204 and that include the unique ID, are captured by the Carrier Partner 204 and routed to the communications platform 202. Thus, any communications technology that the communications platform is servicing, such as calls, messages, delivery to CRM, video conferences, etc., are captured by the Carrier Partner 204 and once the unique identifier is recognized, the communications are routed to the communications platform 202. In the illustrated example, the provisioning by the Carrier Partner 204 can be accomplished by invoking one or more particular API proxy functions, such as the three listed in proxy function block 250, namely API:/organizations/, API:/admins/, and API:/ptns/.

One of the features or operations of the communications platform 202 is the provision and/or orchestration of archival services. The archival services can be provided by the communications platform 202 as messages or communications are received from the Carrier Partner 204 or directly by the communications platform 202. For communications received by the communications platform 202, the communications platform 202 may store directly into enterprise archive 260 or may send to a message archive 242 function provided by the Carrier Partner 204. For communications received by the Carrier Partner 204, the Carrier Partner 204 can either provide to the communications platform 202 for storage within the enterprise archive 260 by means of an API call 252 (i.e., API:/archive/) to the API 222 of the communications platform 202. Alternatively, the Carrier Partner may transmit the messages to be archived to the communications platform 202 by means of an SMPP transmission. Further, rather than sending messages to storage by the communications platform 202, the Carrier Partner 204 may archive the messages directly and simply forward notification of the same to the communications platform 202. Further, the Carrier Partner 204 may utilize a distributed archival system that can be accessed by other carrier partners and/or the communications platform 202. Further, in some embodiments, the Carrier Partner 204 and the communications platform 202 may maintained duplicate and redundant archives of all communications.

As previously described, one of the functions or features of the communications platform 202 is the ability to provide seamless integration of a wide variety of EUDs, and ultimately to be able to provide seamless integration to all EUDs. This is a very powerful and dynamic feature of the communications platform. In essence, the communications platform can be viewed as the communications unifier overcoming the inability to communicate across diverse channels and languages (or communication techniques protocols, etc.). This capability if provided through the communications platform 202, and in some circumstances with cooperation from the Carrier Partner 204. For instance, the communications platform 202 may directly receive, process and direct communications between EUDs, or may receive communications directed to the communications platform 202 from the Carrier Partner 204. In this latter scenario, a message service 244 of the Carrier Partner 204 receives messages that include the unique ID, then then invokes transmission or forward of the same to the communications platform by issuing an SMPP or MM4 transfer 254 through and SMPP/MM4 interface 224 of the communications platform 202. Likewise, the core/SBC function 246 of the Carrier Partner 204 can receive calls that are associated with the unique ID and then forward the calls to the communications platform 202 via dedicated links between the Carrier Partner 204 and the SBC 256 interface 226 of the communications platform 202. The dedicated lines 256 enable communications to be exchanged securely and reliably between the Carrier Partner 204 and the communications platform 202 with minimal latency.

An SBC (or session border controller) is a network element deployed to protect Session Initiation Protocol (SIP) based communications occurring over the Internet Protocol, such as voice over Internet Protocol (VOIP) as a non-limiting example. Early deployments of SBCs were focused on the borders between two service provider networks in a peering environment. This role has now expanded to include significant deployments between a service provider's access network and a backbone network to provide service to residential and/or enterprise customers.

The term “session” refers to a communication between two or more parties—in the context of telephony, this would be a call. Each call consists of one or more call signaling message exchanges that control the call, and one or more call media streams which carry the call's audio, video, or other data along with information of call statistics and quality. Together, these streams make up a session. It is the job of a session border controller to exert influence over the data flows of sessions.

The term “border” refers to a point of demarcation between one part of a network and another. As a simple example, at the edge of a corporate network, a firewall demarcates the local network (inside the corporation) from the rest of the Internet (outside the corporation). A more complex example is that of a large corporation where different departments have security needs for each location and perhaps for each kind of data. In this case, filtering routers or other network elements are used to control the flow of data streams. It is the job of a session border controller to assist policy administrators in managing the flow of session data across these borders.

The term “controller” refers to the influence that session border controllers have on the data streams that comprise sessions, as they traverse borders between one part of a network and another. Additionally, session border controllers often provide measurement, access control, and data conversion facilities for the calls they control.

SBCs commonly maintain full session state and offer the functions such as:
security to protect the network and other devices from malicious attacks (i.e. denial of service), toll fraud via rogue media streams, malformed packet protection, and encryption of signaling and media;
connectivity to allow different parts of the network to communicate through the use of a variety of techniques including NAT traversal, SIP normalization via SIP message and header manipulation, IPv4 to IPv6 interworking, VPN connectivity and Protocol translations between SIP, SIP-I, H.323;
quality of service (QoS) policy of a network and prioritization of flows including traffic policing, resource allocation, rate limiting, call admission control, and ToS/DSCP bit setting;
regulatory support such as emergency call prioritization and lawful interception;
media services though built-in digital signal processors (DSPs) to enable them to offer border-based media control and services such as DTMF relay and interworking, media transcoding, tones and announcements, data and fax interworking, support for voice and video calls; and
statistics and billing information because all sessions that pass through the edge of the network pass through the SBC, it is a natural point to gather statistics and usage-based information on these sessions.

SBCs are inserted into the signaling and/or media paths between calling and called parties (eg. Within a VoIP call), predominantly those using the Session Initiation Protocol (SIP), H.323, and MGCP call-signaling protocols.

In many cases the SBC hides the network topology and protects the service provider or enterprise packet networks. The SBC terminates an inbound call and initiates the second call leg to the destination party. The effect of this behavior is that not only the signaling traffic, but also the media traffic (voice, video) is controlled by the SBC. In cases where the SBC does not have the capability to provide media services, SBCs are also able to redirect media traffic to a different element elsewhere in the network, for recording, generation of music-on-hold, or other media-related purposes. Conversely, without an SBC, the media traffic travels directly between the endpoints, without the in-network call signaling elements having control over their path.

In other cases, the SBC simply modifies the stream of call control (signaling) data involved in each call, perhaps limiting the kinds of calls that can be conducted, changing the codec choices, and so on. Ultimately, SBCs allow the network operators to manage the calls that are made on their networks, fix or change protocols and protocol syntax to achieve interoperability, and also overcome some of the problems that firewalls and network address translators (NATs) present for VoIP calls.

To show the operation of an SBC, one can compare a simple call establishment sequence with a call establishment sequence with an SBC. In the simplest session establishment sequence with only one proxy between the user agents the proxy's task is to identify the callee's location and forward the request to it. The proxy also adds a Via header with its own address to indicate the path that the response should traverse. The proxy does not change any dialog identification information present in the message such as the tag in the From header, the Call-Id or the Cseq. Proxies also do not alter any information in the SIP message bodies. Note that during the session initiation phase the user agents exchange SIP messages with the SDP bodies that include addresses at which the agents expect the media traffic. After successfully finishing the session initiation phase the user agents can exchange the media traffic directly between each other without the involvement of the proxy.

SBCs are designed for many applications and are used by operators and enterprises to achieve a variety of goals. Even the same SBC implementation might act differently depending on its configuration and the use case. Hence, it is not easily possible to describe an exact SBC behavior that would apply to all SBC implementations. In general, it is possible to identify certain features that are common to SBCs. For example, most SBCs are implemented as back-to-back user agent. A proxy-like server can split an SIP transaction in two call legs: on the side facing the user agent client (UAC), it acts as server, on the side facing user agent server (UAS) it acts as a client. While a proxy usually keeps only state information related to active transactions, it may keep state information about active dialogs, e.g., calls. That is, once a proxy receives a SIP request it will save some state information. Once the transaction is over, e.g., after receiving a response, the state information will soon after be deleted. The state information may be maintained for active calls and only deleted once the call is terminated.

When an SBC is included in the call path, the SBC operates as a user agent server towards the caller and as user agent client towards the callee. In this sense, the SBC actually terminates that call that was generated by the caller and starts a new call towards the caller. The INVITE message sent by the SBC no longer may contain a clear reference to the caller. The INVITE sent by the SBC to the proxy includes Via and Contact headers that point to the SBC itself and not the caller. SBCs often also manipulate the dialog identification information listed in the Call-Id and From tag. Further, in case the SBC is configured to also control the media traffic then the SBC also changes the media addressing information included in the c and m lines of the SDP body. Thereby, not only will all SIP messages traverse the SBC but also all audio and video packets. As the INVITE sent by the SBC establishes a new dialog, the SBC also manipulates the message sequence number (CSeq) as well the Max-Forwards value. Note that the list of header manipulations listed here is only a subset of the possible changes that an SBC might introduce to a SIP message. Furthermore, some SBCs might not do all of the listed manipulations. If the SBC is not expected to control the media traffic then there might be no need to change anything in the SDP body. Some SBCs do not change the dialog identification information and others might even not change the addressing information.

SBCs are often used by corporations along with firewalls and intrusion prevention systems (IPS) to enable calls (eg., VoIP calls) to and from a protected enterprise network. Call service providers use SBCs to allow the use of particular protocols from private networks with Internet connections using NAT, and also to implement strong security measures that are necessary to maintain a high quality of service. SBCs also replace the function of application-level gateways. In larger enterprises, SBCs can also be used in conjunction with SIP trunks to provide call control and make routing/policy decisions on how calls are routed through the LAN/WAN. There are often tremendous cost savings associated with routing traffic through the internal IP networks of an enterprise, rather than routing calls through a traditional circuit-switched phone network.

Additionally, some SBCs can allow calls to be set up between two phones using different signaling protocols (e.g., SIP, H.323, Megaco/MGCP) as well as performing transcoding of the media stream when different codecs are in use. Most SBCs also provide firewall features for call traffic (denial of service protection, call filtering, bandwidth management). Protocol normalization and header manipulation is also commonly provided by SBCs, enabling communication between different vendors and networks.

In the illustrated example of FIG. 2, the communications platform 202 is shown as interfacing with Microsoft Teams 270. It will be appreciated that the exemplary architecture and interface with any of a wide variety of applications and/or EUDs such as Microsoft Teams and the use of Microsoft Teams is simply provided as an example. The communications platform 101 interfaces to a Microsoft Teams SBC 272 for voice and/or video content while messages and data can be provided through an MLDTeams application 274. Messages received from the Carrier Partner 204 are placed into a transaction queue 228 and the content 230 can then be transferred to Microsoft Teams 270 via the MLDTeams interface 274.

The architecture illustrated in FIG. 2 also enables FCC compliance, Legal Intercept Compliance, and Emergency Communications Compliance.

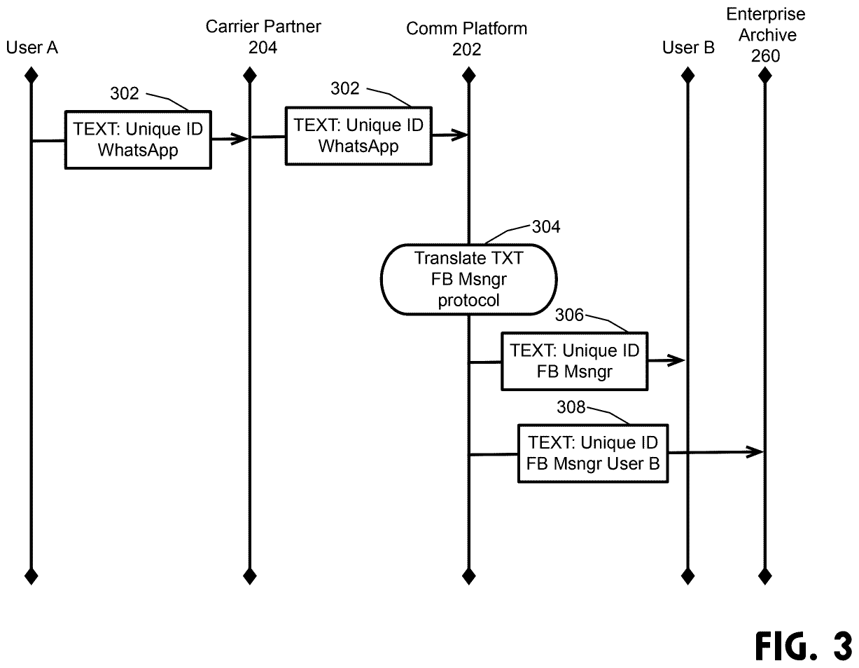

A few practical examples of the operation of the architecture illustrated in FIG. 2 are presented. FIG. 3 is a flow diagram illustrating a first communication exchange example based on an exemplary architecture such as that illustrated in FIG. 2 as a non-limiting example. In this example, assume that User A utilizes WhatsApp to talk with User B who uses FB Messenger and that User B is utilizing an EUD that subscribe to the integrated communications service provide by the communications platform 202. Initially, User A sends a text 302 to User B, the text includes the destination of User B. The Carrier Partner 204 sees the User B destination and determines that it is to be processed by the communications platform 202. As such, the Carrier Partner 204 forwards the message 302 to communications platform 202. The communications platform then identifies the destination of the message and determines that the message is directed to a particular EUD with a particular unique ID and that has a preference known to the communications platform 202 of using FACEBOOK MESSENGER for exchanging of texts messages. The communications platform 202 then performs a protocol translation of the message from WHATSAPP to FACEBOOK MESSENGER 304 and forwards the message to the intended destination 306—User B. The communications platform 202 may also record the message into the Enterprise Archive 260 for User B in FACEBOOK MESSENGER format 308 tied to the unique ID.

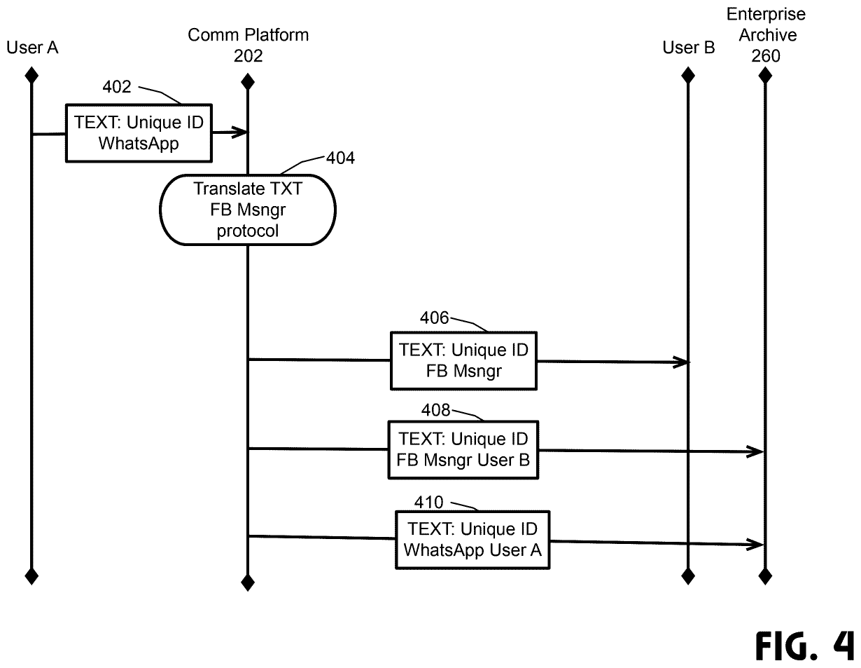

FIG. 4 is a flow diagram illustrating a communication exchange example based on an exemplary architecture such as that illustrated in FIG. 2. In this example, assume that User A utilizes WhatsApp to talk with User B who uses FB Messenger and that both User A and User B are utilizing EUDs that subscribe to the integrated communications service provide by the communications platform 202. Initially, User A sends a text 402 to User B, the text includes the unique ID of User A and the destination of User B. The text 402 is received by the communications platform 202. The communications platform 202 then identifies the destination of the message and determines that the message is directed to a particular EUD what has a preference of using FACEBOOK MESSENGER for exchanging of texts messages. The communications platform 202 then performs a protocol translation of the message from WHATSAPP to FACEBOOK MESSENGER 404 and forwards the message to the intended destination 406—User B using the unique ID of User B. Communications platform 202 may employ the MultiLine/multichannel technology presented in U.S. Pat. Nos. 11,563,711 and/or 9,332,128 (both of which are incorporated herein above by reference) in sending the communications between User A and User B. The communications platform 202 may also record the message into the Enterprise Archive 260 for User B in FACEBOOK MESSENGER format 408 and User A in WHATSAPP format 410.

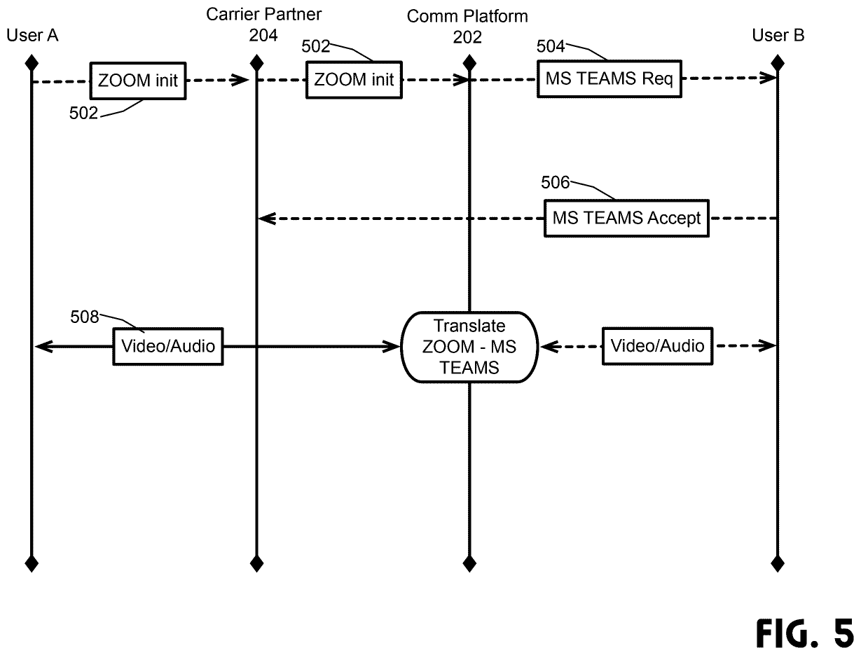

FIG. 5 is a flow diagram illustrating another communication exchange example based on an exemplary architecture such as that illustrated in FIG. 2. User A and User B enter a video conference with User A using ZOOM and User B using MICROSOFT TEAMS. The video conference is initially set up with User A initiating the conference with User B 502. During the setup process, the Carrier Partner 204 detects the unique ID of User A in setting up the conference call and forwards the setup request to the communication platform 202 to be processed. The communication platform 202 recognizes the unique ID for User A and the unique ID of User B and thus identifies both as subscribers to the service. The communication platform 202 then identifies that User B prefers MICROSOFT TEAMS and that User A prefers to use ZOOM. The communications platform 202 then sends an MS TEAMS request 504 to User B. Upon receiving an MS TEAMS accept 506 from User B the communication platform 202 completes the video call connection between User A and User B. Video and Audio between User A and the communication platform is in the ZOOM format and between the communication platform 202 and User B is in MICROSOFT TEAMS format 510. The communication platform 202 performs the translations as well as providing translations for messages transmitted between the two users, archived deposits, etc.

Thus, it should be appreciated that call or message communication happens through a carrier powered endpoint (mobile phone, tablet, connected device) and then that call arrives to the communication platform with a single unique identity. With that single unique identity, the communication platform can do several things using the MultiLine/multichannel technology presented in U.S. Pat. No. 9,332,128 for delivery to different end points (CRM, ZOOM, TEAMS, Messaging systems) and also to get the carrier to terminate as needed and appropriate.

What is important to various embodiments of the present invention is the provision of the single unique identifier from the EUD to invoke the provision of the integrated communication services by the communication platform. The single unique identifier is provided in various manners dependent upon the technology or protocol being employed during the communication setup. In general, it will be understood that the integrated communication services can be accessed by any EUD that is provisioned to operate over a carrier network or, any application or system that operates on such an EUD. As those skilled in the art will understand, a carrier provisioned EUD will include a Subscriber Identity Module (“SIM”) card or an Embedded Subscriber Identity Module (“ESIM”). The SIM card or ESIM is provisioned by the carrier and is unique to the EUD. When the EUD is utilized to set up a communication, such as placing a call, sending a text, sending and SMS, MMS, establishing a video conference, etc., the EUD must first transmit a setup request and that request will include the single unique identifier associated with the EUD or user of the EUD. The single unique identifier may be provisioned to the EUD by the operator of the communication platform or may be provisioned by the carrier in response to the operator of the EUD requesting such provisioning.

Under each of the cellular protocols, during a call setup request, the EUD transmits identifying information as to the originating party or unit. This is most typically in the form of calling line identifier (“CLID”); however, it should be appreciated that many protocols include additional space above and beyond just space in the protocol for the 10-digit phone number associated with the initiating device. As such, during a typical call setup, the EUD may provide the mobile identification number (“MIN”), unique SIM information, the network number that the EUD is provisioned with, as well as additional unique identification information. This information is provisioned into the EUD device by a carrier partner when the user is subscribing to the integrated communication services. This single unique identification number is provided to the carrier operator during all communication set up initiations and, the carrier operator receives the single unique identification number and forwards the communication setup request to the communications platform for processing as described above.

Certain actions or blocks in the processes or process flows described in this specification naturally precede others for the embodiment to function as described. However, the various embodiments are not limited to the order of the actions or blocks as presented or described. That is, it is recognized that some actions or blocks may be performed before, after, or in parallel (substantially simultaneously with) other actions or blocks without departing from the scope and spirit of the various embodiments. In some embodiments, certain actions or blocks may be omitted or not performed as not all embodiments necessarily must implement all of the described actions. Also, in some embodiments, multiple actions depicted and described as unique actions or blocks in the present disclosure may be comprised within a single step or block. Further, words such as “thereafter”, “then”, “next”, “subsequently”, etc. are not intended to limit the order of the actions or blocks. These words are simply used to guide the reader through the description of the exemplary method.

Additionally, one of ordinary skill in programming will be able to write computer code or identify appropriate hardware and/or circuits to implement the various embodiments, as well as features and aspects thereof, based on the flow charts and associated description in this specification. Therefore, disclosure of a particular set of program code instructions or detailed hardware devices is not considered necessary for an adequate understanding of how to make and use the various embodiments. The functionality of the claimed computer implemented processes is explained in more detail in the above description and in conjunction with the Figures that may illustrate various process flows.

Based on all of the above, as those of ordinary skill in the art would understand, under existing traditional technology the network services and identifiers have remained siloed and network specific, they were not unique across ecosystems. The uniqueness e of various embodiments of the present invention utilize a single identifier (an MSISDN, a SIM, an ESIM, or a uniquely asserted self-sovereign identity) to now bring all channels of communication and fragmented technologies together. Similar to the reversal of the Tower of Babel or, the user of a Babel Fish in the car as presented in Hitch Hikers Guide to the Galaxy.

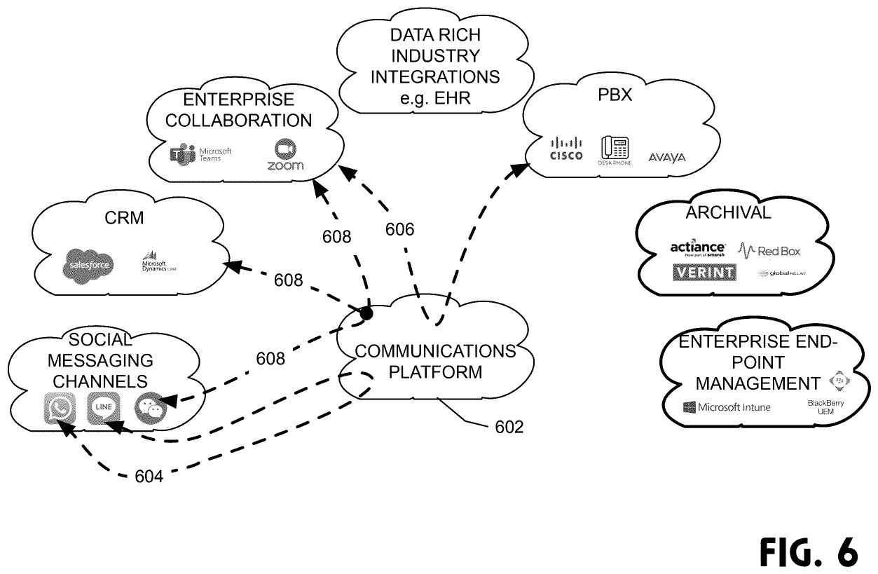

FIG. 6 is a sky of clouds illustrating the overall operation of an embodiment of an exemplary communications platform 602. The communications platform operates as the Babel Fish and enables communication between all of the various platforms, technologies, communications channels, terminal devices, communication applications, etc. A single unique identity is provided for each user, or each position, etc. This single unique identity is what the communications platform 602 utilizes to recognize a communications initiator and a communications recipient.

The communications platform 602 is shown as effectively operating in the middle of a complex orchestration of communication. A few scenarios are illustrated in FIG. 6. For instance, communication session 604 is between a first EUD using the WHATSAPP application and another EUD using the LINE messaging app. In this scenario, assume the communication was initiated by the user of the WHATSAPP application. That user initiates a connection to the user of the LINE messaging app. The communications platform 602 receives the communication initiation, either directly or as being recognized by the communications infrastructure (i.e. PSTN, MTN, etc), and recognizes the unique identification of the initiating EUD. The communications platform 602 also recognizes the destination and the unique identifier associated with the destination EUD. The communications platform 602 thus knows that the initiating EUD is communicating with the WHATSAPP application and the receiving EUD is communicating with the LINE messaging application. The communications platform 602 can perform any necessary translations on the communication between the two EUDs and store a history of the communications session into the archives for both EUDs.

As another example, communication session 606 is between an initiating EUD utilizing a ZOOM connection and receiving EUD utilizing a PBX connection. The communications platform 602 receives the communication initiation from the initiating EUD and recognizes the unique identifier associated therewith. The communications platform 602 also recognizes the unique identifier of the receiving EUD. The communications platform 602 operates as a bridge to enable these two disparate communications technologies to actually be engaged in a communications session. The communications platform 602 receives voice communications from the ZOOM app and converts it as necessary to send to the PBX and vice versa. The communications platform 602 is aware that the PBX cannot provide video or process video and as such, simply obtains and provides the voice communications from the ZOOM app. The communications platform 602 can also provide an indicator that the receiving EUD is not video capable by providing such information as a ZOOM avatar for the receiving EUD.

As another example, communication session 608 illustrates a one-to-many communications session established by a messaging app EUD with a MICROSOFT TEAMS EUD and a MICROSOFT DYNAMICS CRM EUD. Similarly, the communications platform recognizes the unique identifiers, identifies a language or protocol that can I be spoken to each EUD and coordinates the communication session.

Thus, it should be appreciated that the communications platform 602, utilizing the single unique identifiers for each EUD, can establish a communications session between any technology and breaks down the silos imposed by current state of the art technology.

In one or more exemplary aspects, the functions described may be implemented in hardware, software, firmware, or any combination thereof. If implemented in software, the functions may be stored on or transmitted as one or more instructions or code on a computer-readable medium. Computer-readable media include both computer storage media and communication media including any medium that facilitates transfer of a computer program from one place to another.

In the description and claims of the present application, each of the verbs, “comprise”, “include” and “have”, and conjugates thereof, are used to indicate that the object or objects of the verb are not necessarily a complete listing of members, components, elements, or parts of the subject or subjects of the verb.

A storage media may be any available media that may be accessed by a computer. By way of example, and not limitation, such computer-readable media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer.

Also, any connection is properly termed a computer-readable medium. For example, if the software is transmitted from a website, server, or other remote source using a coaxial cable, fiber optic cable, twisted pair, digital subscriber line (“DSL”), or wireless technologies such as infrared, radio, and microwave, then the coaxial cable, fiber optic cable, twisted pair, DSL, or wireless technologies such as infrared, radio, acoustic and microwave are included in the definition of medium.

Disk and disc, as used herein, includes compact disc (“CD”), laser disc, optical disc, digital versatile disc (“DVD”), floppy disk and blu-ray disc where disks usually reproduce data magnetically, while discs reproduce data optically with lasers. Combinations of the above should also be included within the scope of computer-readable media.

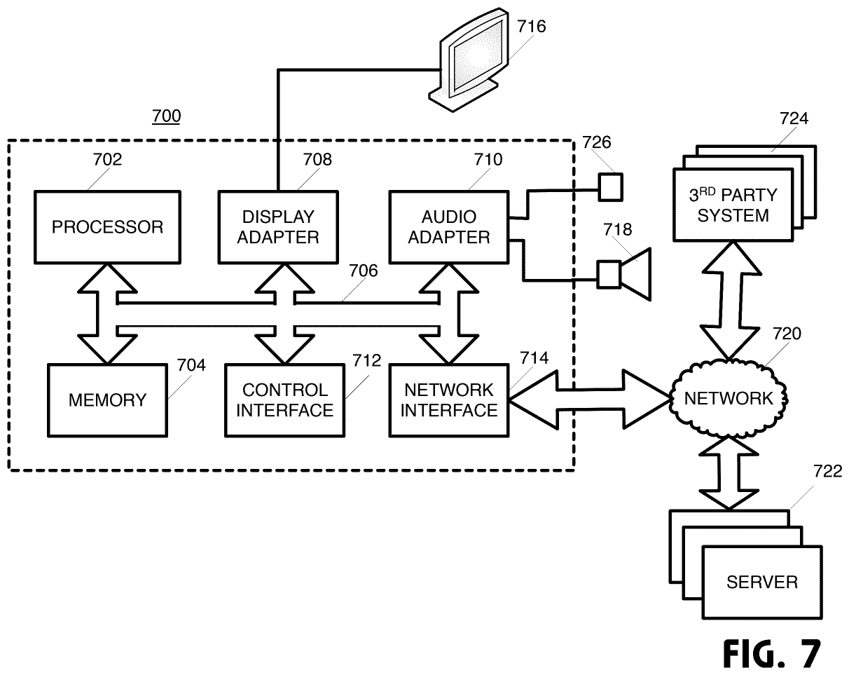

FIG. 7 is a functional block diagram of the components of an exemplary embodiment of system or sub-system operating as a controller or processor 700 that could be used in various embodiments of the disclosure for controlling aspects of the various embodiments. It will be appreciated that not all of the components illustrated in FIG. 7 are required in all embodiments of the activity monitor but, each of the components are presented and described in conjunction with FIG. 7 to provide a complete and overall understanding of the components. The controller can include a general computing platform 700 illustrated as including a processor/memory device 702/704 that may be integrated with each other or, communicatively connected over a bus or similar interface 706. The processor 702 can be a variety of processor types including microprocessors, micro-controllers, programmable arrays, custom IC's etc. and may also include single or multiple processors with or without accelerators or the like. The memory element of 704 may include a variety of structures, including but not limited to RAM, ROM, magnetic media, optical media, bubble memory, FLASH memory, EPROM, EEPROM, etc. The processor 702, or other components in the controller may also provide components such as a real-time clock, analog to digital convertors, digital to analog convertors, etc. The processor 702 also interfaces to a variety of elements including a control interface 712, a display adapter 708, an audio adapter 710, and network/device interface 714. The control interface 712 provides an interface to external controls, such as sensors, actuators, drawing heads, nozzles, cartridges, pressure actuators, leading mechanism, drums, step motors, a keyboard, a mouse, a pin pad, an audio activated device, as well as a variety of the many other available input and output devices or, another computer or processing device or the like. The display adapter 708 can be used to drive a variety of alert elements 716, such as display devices including an LED display, LCD display, one or more LEDs or other display devices. The audio adapter 710 interfaces to and drives another alert element 718, such as a speaker or speaker system, buzzer, bell, etc. The network/interface 714 may interface to a network 720 which may be any type of network including, but not limited to the Internet, a global network, a wide area network, a local area network, a wired network, a wireless network or any other network type including hybrids. Through the network 720, or even directly, the controller 700 can interface to other devices or computing platforms such as one or more servers 722 and/or third party systems 724. A battery or power source provides power for the controller 700.

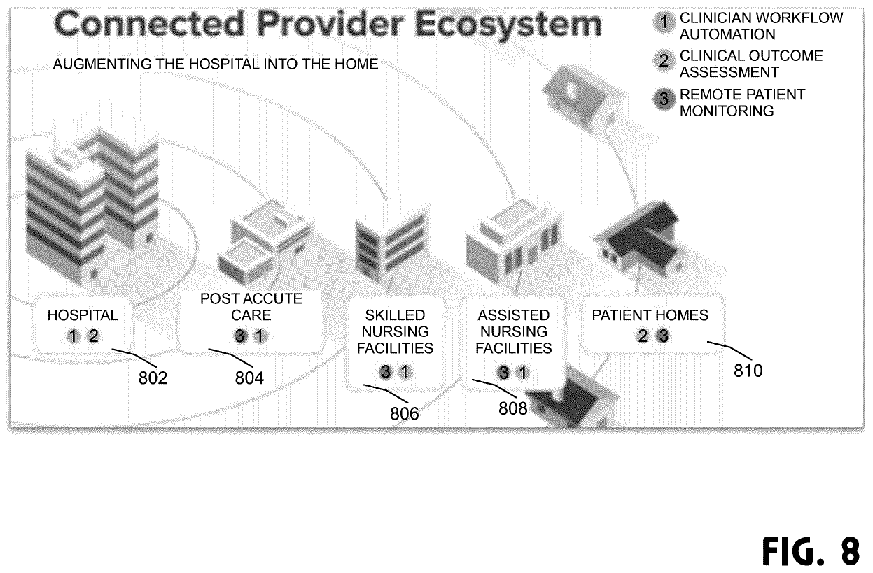

FIG. 8 is a conceptual diagram illustrating the connected provider ecosystem that can be achieved through deployment of embodiments of the present invention. Three types of activities are identified: (1) clinician workflow automation; (2) clinical outcome assessment; and (3) remote patient monitoring. The distribution of services is shown with concentric circles with the hospital 802 being at the heart of the service center, followed by post acute care 804, skilled nursing facilities 806, assisted nursing facilities 808, and finally patient homes 810 at the extremity of the concentric circles. The conceptual diagram illustrates that embodiments of the invention enable cross communication and data sharing across all sectors with the provision of necessary and applicable services at each location.

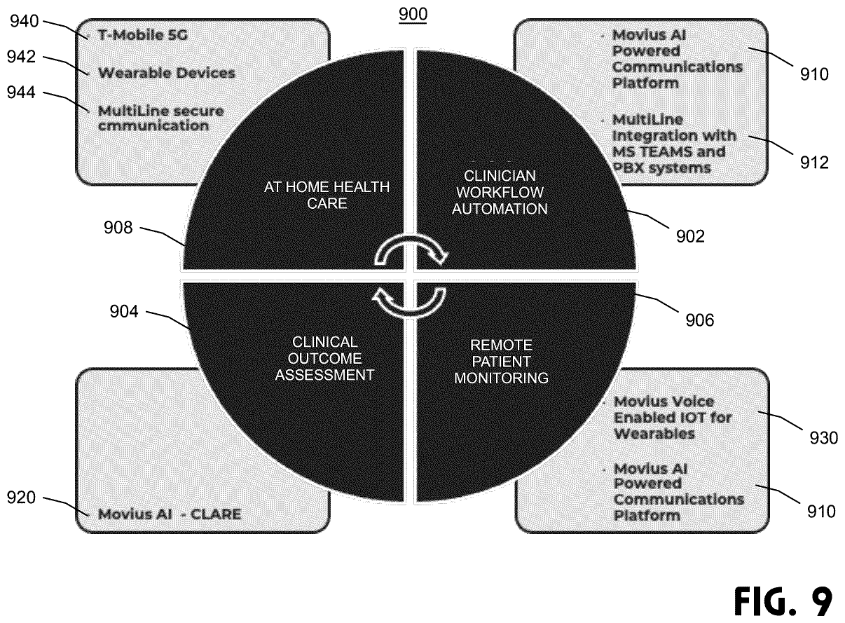

FIG. 9 is a conceptual diagram illustrating a summary of the solutions provided by the various embodiments of the present invention. Four types of activities are identified: clinician workflow automation 902; clinical outcome assessment 904; remote patient monitoring 906; and at home health care 908. The activities illustrated in the flow cycle 900 are implemented and/or supported by various aspects of the CRAC. The clinician workflow automation 902 is shown as drawing support from an AI powered communications platform 910, such as the platform provided by MOVIUS INTERACTIVE CORPORATION and employment of the MultiLine integration with MICROSOFT TEAMS and a private branch exchange (PBX) system 912.

The Clinical outcome assessment 904 is shown as drawing support from an AI system 920 such as CLARE developed by MOVIUS INTERACTIVE CORPORATION as a non-limiting example. So equipped, the clinician workflow automation provides enhanced documentation quality and efficiency, patient updates through multi-channel voice and messaging, and EHR/EMR integrations. The use cases enabled by the clinical outcome assessment 904 includes transcription, topic analysis, sentiment and emotion analysis. Further, it provides aggregated reporting and insights as well as multi-lingual support.

Remote patient monitoring 906 draws upon the use of voice enabled IOT for wearables 930 that can be used to monitor patient activity, stats, etc. The voice enabled IOT 930, such as the technology developed by MOVIUS INTERACTIVE CORPORATION and presented in U.S. Pat. No. 11,032,427 (incorporated herein above by reference) as well as the AI powered communications platform 910. The use cases provided with remote patient monitoring 906 include integration of MultiLine with wearable devices and providing voice channels to IOT enabled devices, and the provision of an AI platform for aggregation and insights.

At home health care 908 draws from the use of mobile devices 940, such as a T-MOBILE 5G device as a non-limiting example, wearable devices 942, and MultiLine secure communication 944. With the at home health care service 908, the use cases provided include separation of personal and professional lines for care providers, the ability to engage with patients and medical support anytime and anywhere, as well as emergency response.

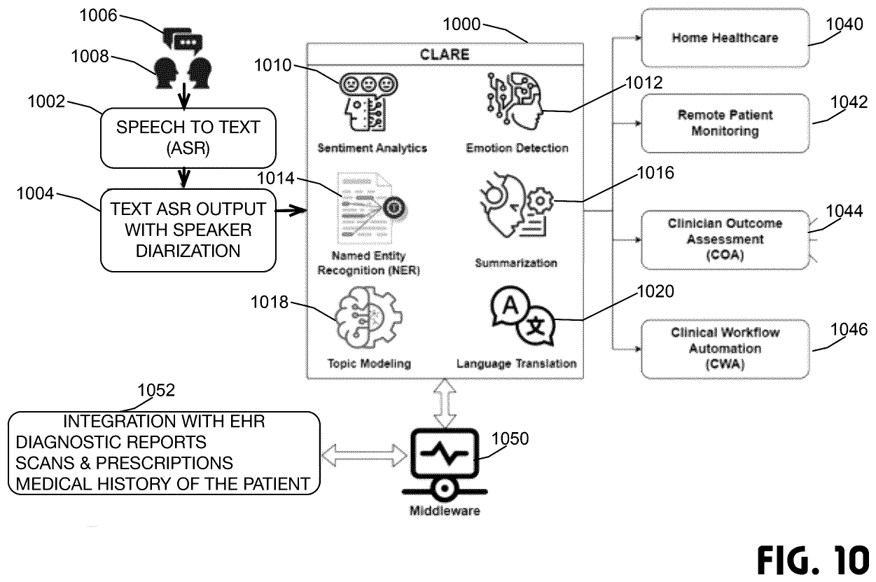

FIG. 10 is a conceptual block diagram illustrating the use of an AI tool within various embodiments of the CRAC. It should be appreciated that several types of AI tools can be employed by embodiments of the CRAC and that not all functions described herein are required nor is the AI tool limited to the disclosed functions. For purposes of illustration, the AI tool will be described based on the CLARE AI tool developed by MOVIUS INTERACTIVE CORPORATION. As previously described, on aspect of the CRAC is the ability to automatically collect discussions between a healthcare provider and a patient or another healthcare provider and make those discussions storable and retrievable. Because typing is time consuming and prone to error, embodiments of the CRAC may employ the use of speech-to-text conversion 1002. Thus, using the healthcare providers device, a conversation 1006 between two or more entities 1008 is captured by the Automatic Speech to Text (ASR) function. It should be appreciated that the ASR function may be an app and/or hardware residing on the provider's device or it may be a separate software and/or hardware device, program, module, system, etc. that then provides the text output to the next stage in the process. As such, the healthcare providers device may receive the analog voice and perform the ASR function or pass the voice to another device for ASR or, some other device may receive the analog voice and perform the ASR and provide the text to the provider's device or some other device in preparation for presentment to the AI tool 1000.

The output of the ASR 1002 is then provided to another functional block 1004 for speaker diarization processing. Those skilled in the relevant art will appreciate that speaker diarization is the process of partitioning an audio stream containing human speech into homogeneous segments according to the identity of each speaker. It can enhance the readability of an automatic speech transcription by structuring the audio stream into speaker turns and, when used together with speaker recognition systems, by providing the speaker's identity. It should be appreciated that the speaker diarization may be performed on the raw audio voice or it may be performed on the text output from an ASR or a combination of both. In speaker diarization, one of the most popular methods is to use a Gaussian mixture model to model each of the speakers, and assign the corresponding frames for each speaker with the help of a Hidden Markov Model. There are two main kinds of clustering strategies. The first one is by far the most popular and is called Bottom-Up. The algorithm starts in splitting the full audio content in a succession of clusters and progressively tries to merge the redundant clusters in order to reach a situation where each cluster corresponds to a real speaker. The second clustering strategy is called top-down and starts with one single cluster for all the audio data and tries to split it iteratively until reaching a number of clusters equal to the number of speakers. Speaker diarization may also be performed via neural networks leveraging large-scale GPU computing and methodological developments in deep learning.

Once speaker diarization and ASR has been conducted, the diarized and text converted data is provide to the AI tool 1000 for processing. In the illustrated embodiment the CLARE AI tool 1000 conducts one or more of the functions including sentiment analytics 1010, emotion detection 1012, named entity recognition (NER) 1014, summarization 1016, topic modeling 1018, and language translation 1020.

Sentiment analytics is opinion mining or emotion AI. Sentiment analytics may utilize natural language processing, text analysis, computational linguistics, and biometrics to systematically identify, extract, quantify, and study affective states and subjective information. Sentiment analysis is widely applied to voice of the customer materials such as reviews and survey responses, online and social media, and healthcare materials for applications that range from marketing to customer service to clinical medicine. With the rise of deep language models, such as ROBERTa, also more difficult data domains can be analyzed, e.g., news texts where authors typically express their opinion/sentiment less explicitly.

A basic task in sentiment analysis is classifying the polarity of a given text at the document, sentence, or feature/aspect level-whether the expressed opinion in a document, a sentence or an entity feature/aspect is positive, negative, or neutral. Advanced, “beyond polarity” sentiment classification looks, for instance, at emotional states such as enjoyment, anger, disgust, sadness, fear, and surprise.

Sentimental analysis may include quantifying patterns in text and, separately, psychological research that examined a person's psychological state based on analysis of their verbal behavior. Some systems operate to identify and correlate individual words and phrases in text with respect to different emotional scales. For instance, synonyms or related words may be ranked based on an emotional weighting for the word. Such as hate would be weighted differently than dislike.

However, some systems may simply employ a polar view of sentiment, from positive to negative while some techniques may employ the use of a neutral as well. Even though in most statistical classification methods, the neutral class is ignored under the assumption that neutral texts lie near the boundary of the binary classifier, several researchers suggest that, as in every polarity problem, three categories must be identified. There are in principle two ways for operating with a neutral class. Either the algorithm proceeds by first identifying the neutral language, filtering it out and then assessing the rest in terms of positive and negative sentiments, or it builds a three-way classification in one step. This second approach often involves estimating a probability distribution over all categories (e.g. naive Bayes classifiers as implemented by the NLTK). Whether and how to use a neutral class depends on the nature of the data: if the data is clearly clustered into neutral, negative and positive language, it makes sense to filter the neutral language out and focus on the polarity between positive and negative sentiments. If, in contrast, the data are mostly neutral with small deviations towards positive and negative affect, this strategy would make it harder to clearly distinguish between the two poles.

A different method for determining sentiment is the use of a scaling system whereby words commonly associated with having a negative, neutral, or positive sentiment with them are given an associated number on a −10 to +10 scale (most negative up to most positive) or simply from 0 to a positive upper limit such as +4. This makes it possible to adjust the sentiment of a given term relative to its environment (usually on the level of the sentence). When a piece of unstructured text is analyzed using natural language processing, each concept in the specified environment is given a score based on the way sentiment words relate to the concept and its associated score. This allows movement to a more sophisticated understanding of sentiment, because it is now possible to adjust the sentiment value of a concept relative to modifications that may surround it. Words, for example, that intensify, relax or negate the sentiment expressed by the concept can affect its score. Alternatively, texts can be given a positive and negative sentiment strength score if the goal is to determine the sentiment in a text rather than the overall polarity and strength of the text.

There are various other types of sentiment analysis, such as aspect-based sentiment analysis, grading sentiment analysis (positive, negative, neutral), multilingual sentiment analysis and detection of emotions. All of the above-described operations or techniques, as well as other could be employed in the AI tool.

Emotion detection or recognition is the process of identifying human emotion. People vary widely in their accuracy at recognizing the emotions of others. Use of technology to help people with emotion recognition is a relatively nascent research area. Generally, the technology works best if it uses multiple modalities in context. In some systems, the detection is augmented by automating the recognition of facial expressions from video, spoken expressions from audio, written expressions from text, and physiology as measured by wearables.

Humans show a great deal of variability in their abilities to recognize emotion and thus, it should be understood that there are several sources of “ground truth”, or truth about what the real emotion is. Suppose we are trying to recognize the emotions of person A. One source is “what would most people say that person A is feeling?” In this case, the ‘truth’ may not correspond to what person A feels, but may correspond to what most people would say it looks like person feels. For example, person A may actually feel sad, but he puts on a big smile and then most people say he looks happy. If an automated method achieves the same results as a group of observers it may be considered accurate, even if it does not actually measure what person A truly feels. Another source of ‘truth’ is to ask person A what he or she truly feels. This works if person A has a good sense of his or her internal state, and wants to tell admit what it is, and is capable of putting it accurately into words or a number. However, some people are alexithymic and do not have a good sense of their internal feelings, or they are not able to communicate them accurately with words and numbers. In general, getting to the truth of what emotion is actually present can take some work, can vary depending on the criteria that are selected, and will usually involve maintaining some level of uncertainty.

In some embodiments, the analysis of human expressions from multimodal forms such as texts, physiology, audio, or video can be utilized to improve the accuracy of emotion recognition. Different emotion types can be detected through the integration of information from facial expressions, body movement and gestures, and speech.

The existing approaches in emotion recognition to classify certain emotion types can be generally classified into three main categories: knowledge-based techniques, statistical methods, and hybrid approaches.

Various embodiments of the CRAC may also include gender, race and age identification. The AI tool may include algorithms to not only detect certain characteristics of the voice for performing this, but also parsing the text to identify words, phrases and speech patterns that may be relevant to different genders and/or ages. In addition, the use of facial scanning or body scanning can also be beneficial in helping to ascertain gender, race, age, etc. The ability to discern gender, race and age is beneficial for record keeping, analysis and even biometric processing or authentication.

Deep learning is an AI technique that seeks to learn from experience to resemble the human brain. Through a training procedure, these representations are learned. To teach the software how to detect an object, it must first be trained with a large number of object images that are categorized according to different classes. Deep learning-based algorithms, on average, require a large amount of training data and take longer to train than traditional machine learning methods. Finding unique attributes when trying to recognize any object or character on an image is time-consuming and complex. Unlike traditional machine learning, where features are manually retrieved, problems can be solved using deep learning approaches, which extract important characteristics from data automatically. A neural network with multiple hidden layers is known as deep learning. They may build complicated notions from simple concepts after an image has been taught over the network. By integrating simple elements such as shape, edges, and corners, an image can be trained in the network to learn items such as characters, faces, and so on. As the image travels through the layers, each one gets a simple property while moving on to the next. As the layers grow larger, the network may learn more complex features and eventually merge them to identify the image.

A CNN (convolutional neural network) is a kind of artificial neural network that is commonly used for image or object identification and categorization. Using a CNN, Deep Learning recognizes items in an image. An input layer, hidden layers, and an output layer are all part of a standard neural network. The anatomy of the brain inspired CNNs. Artificial neurons or nodes in CNNs collect inputs, process them, and deliver the result as output, rather like a neuron inside the brain functions and transmits signals between cells. The images are used as a source of data. Multiple hidden layers may exist in CNNs, each of which performs feature extraction from the image by performing calculations. The very first layer that extracts feature out of an input image is convolution. The object is classified and identified in the output layer by the fully connected layer. The convolutional layer is the most important constituent of CNN. The mathematical procedure of convolution is used to combine two sources of data. Gender estimation from social image collection, images that do not require access to private details of the subject areas that are not displayed in the images, such as their birth date, and the usual approach that includes the collection of other information about an individual and on the basis about which gender can be ascertained on manually handled annotated data for gender recognition. D-CNN is a technique that works directly on images and aids in precise gender estimation. Overfitting is usually a minor issue. Algorithms or products such as NYCKEL and PYTHON DEEP LEARNING can be utilize to enhance this service.

Knowledge-based techniques (sometimes referred to as lexicon-based techniques), utilize domain knowledge and the semantic and syntactic characteristics of text and potentially spoken language in order to detect certain emotion types. In this approach, it is common to use knowledge-based resources during the emotion classification process such as WORDNE, SENTICNET, CONCEPTNET, and EMOTINET as non-limteing examples. One of the advantages of this approach is the accessibility and economy brought about by the large availability of such knowledge-based resources. A limitation of this technique on the other hand, is its inability to handle concept nuances and complex linguistic rules.

Knowledge-based techniques can be mainly classified into two categories: dictionary-based and corpus-based approaches. Dictionary-based approaches find opinion or emotion seed words in a dictionary and search for their synonyms and antonyms to expand the initial list of opinions or emotions. Corpus-based approaches on the other hand, start with a seed list of opinion or emotion words, and expand the database by finding other words with context-specific characteristics in a large corpus. While corpus-based approaches take into account context, their performance still vary in different domains since a word in one domain can have a different orientation in another domain.

Statistical methods commonly involve the use of different supervised machine learning algorithms in which a large set of annotated data is fed into the algorithms for the system to learn and predict the appropriate emotion types. Machine learning algorithms generally provide more reasonable classification accuracy compared to other approaches, but one of the challenges in achieving good results in the classification process, is the need to have a sufficiently large training set.

Some of the most commonly used machine learning algorithms include Support Vector Machines (SVM), Naive Bayes, and Maximum Entropy. Deep learning, which is under the unsupervised family of machine learning, is also widely employed in emotion recognition. Well-known deep learning algorithms include different architectures of Artificial Neural Network (ANN) such as Convolutional Neural Network (CNN), Long Short-term Memory (LSTM), and Extreme Learning Machine (ELM). The popularity of deep learning approaches in the domain of emotion recognition may be mainly attributed to its success in related applications such as in computer vision, speech recognition, and Natural Language Processing (NLP).

Hybrid approaches in emotion recognition are essentially a combination of knowledge-based techniques and statistical methods, which exploit complementary characteristics from both techniques. Some of the works that have applied an ensemble of knowledge-driven linguistic elements and statistical methods include sentic computing and iFeel, both of which have adopted the concept-level knowledge-based resource SenticNet. The role of such knowledge-based resources in the implementation of hybrid approaches is highly important in the emotion classification process. Since hybrid techniques gain from the benefits offered by both knowledge-based and statistical approaches, they tend to have better classification performance as opposed to employing knowledge-based or statistical methods independently. A downside of using hybrid techniques however, is the computational complexity during the classification process.

Data is an integral part of the existing approaches in emotion recognition and in most cases and as such, obtaining good and broad annotated data is necessary to properly train machine learning algorithms. For the task of classifying different emotion types from multimodal sources in the form of texts, audio, videos or physiological signals, the following datasets are available:
HUMAINE: provides natural clips with emotion words and context labels in multiple modalities;
Belfast database: provides clips with a wide range of emotions from TV programs and interview recordings;
SEMAINE: provides audiovisual recordings between a person and a virtual agent and contains emotion annotations such as angry, happy, fear, disgust, sadness, contempt, and amusement;
IEMOCAP: provides recordings of dyadic sessions between actors and contains emotion annotations such as happiness, anger, sadness, frustration, and neutral state;
eNTERFACE: provides audiovisual recordings of subjects from seven nationalities and contains emotion annotations such as happiness, anger, sadness, surprise, disgust, and fear;
DEAP: provides electroencephalography (EEG), electrocardiogramand face video recordings, as well as emotion annotations in terms of valence, arousal, and dominance of people watching film clips;
DREAMER: provides electroencephalography (EEG) and electrocardiography (ECG) recordings, as well as emotion annotations in terms of valence, dominance of people watching film clips;
MELD: is a multiparty conversational dataset where each utterance is labeled with emotion and sentiment. MELD[28] provides conversations in video format and hence suitable for multimodal emotion recognition and sentiment analysis. MELD is useful for multimodal sentiment analysis and emotion recognition, dialogue systems and emotion recognition in conversations;
MuSe: provides audiovisual recordings of natural interactions between a person and an object. [30] It has discrete and continuous emotion annotations in terms of valence, arousal and trustworthiness as well as speech topics useful for multimodal sentiment analysis and emotion recognition;
UIT-VSMEC: is a standard Vietnamese Social Media Emotion Corpus (UIT-VSMEC) with about 6,927 human-annotated sentences with six emotion labels, contributing to emotion recognition research in Vietnamese which is a low-resource language in Natural Language Processing (NLP); and
BED: provides valence and arousal of people watching images. It also includes electroencephalography (EEG) recordings of people exposed to various stimuli (SSVEP, resting with eyes closed, resting with eyes open, cognitive tasks) for the task of EEG-based biometrics.

Named entity recognition (NER) 1014, is a technique in natural language processing (NLP) that focuses on identifying and classifying entities. The purpose of NER is to automatically extract structured information from unstructured text, enabling machines to understand and categorize entities in a meaningful manner for various applications like text summarization, building knowledge graphs, question answering, and knowledge graph construction.

Name-entity recognition (NER) is also referred to as entity identification, entity chunking, and entity extraction. NER is the component of information extraction that aims to identify and categorize named entities within unstructured text. NER involves the identification of key information in the text and classifies it into a set of predefined categories. An entity is the thing that is consistently talked about or refer to in the text, such as person names, organizations, locations, time expressions, quantities, percentages and more predefined categories.

NER systems find applications across various domains, including question answering, information retrieval and machine translation. NER plays an important role in enhancing the precision of other NLP tasks like part-of-speech tagging and parsing. At its core, NLP is just a two-step process including detecting the entities from the text and classifying them into different categories.

An NER system analyses an input text to identify and locate the named entities. The system then identifies the sentence boundaries by considering capitalization rules. It recognizes the end of the sentence when a word starts with a capital letter, assuming it could be the beginning of a new sentence. Knowing sentence boundaries aids in contextualizing entities within the text, allowing the model to understand relationships and meanings.

NER can be trained to classify entire documents into different types, such as invoices, receipts, or passports. Document classification enhances the versatility of NER, allowing it to adapt its entity recognition based on the specific characteristics and context of different document types.

NER employs machine learning algorithms, including supervised learning, to analyze labeled datasets. These datasets contain examples of annotated entities, guiding the model in recognizing similar entities in new, unseen data. Through multiple training iterations, the model refines its understanding of contextual features, syntactic structures, and entity patterns, continuously improving its accuracy over time. The model's ability to adapt to new data allows it to handle variations in language, context, and entity types, making it more robust and effective.

Various embodiments may employ the use of one or more various NER methods including, but not limited to the following:

Lexicon Based Method. The NER uses a dictionary with a list of words or terms. The process involves checking if any of these words are present in a given text. However, this approach isn't commonly used because it requires constant updating and careful maintenance of the dictionary to stay accurate and effective.

Rule Based Method. The Rule Based NER method uses a set of predefined rules to guide the extraction of information. These rules are based on patterns and context. Pattern-based rules focus on the structure and form of words, looking at their morphological patterns. On the other hand, context-based rules consider the surrounding words or the context in which a word appears within the text document. This combination of pattern-based and context-based rules enhances the precision of information extraction in Named Entity Recognition (NER).

Machine Learning-Based Method. This can include Multi-Class Classification with Machine Learning Algorithms and/or Conditional Random Field (CRF). One way is to train the model for multi-class classification using different machine learning algorithms, but it requires a lot of labelling. In addition to labelling the model also requires a deep understanding of context to deal with the ambiguity of the sentences. This makes it a challenging task for a simple machine learning algorithm.

However, Conditional Random Field (CRF) is implemented by both NLP Speech Tagger and NLTK. It is a probabilistic model that can be used to model sequential data such as words. The CRF can capture a deep understanding of the context of the sentence.

Deep Learning Based Method. Deep learning NER system is much more accurate than previous methods, as it is able to assemble words. This is due to the fact that it uses a method called word embedding, that is capable of understanding the semantic and syntactic relationship between various words. It is also able to analyze topic specific as well as high level words automatically. This makes deep learning NER applicable for performing multiple tasks. Deep learning can do most of the repetitive work itself, hence researchers for example can use their time more efficiently.

Summarization 1016. Text summarization is the process of extracting and piecing together useful information from a long text to form a shorter text that retains the main discussion points or action items, while being easier to read and understand. When summarizing a text, there are two major methods involved:

Extractive summarization: This type of summarization is synonymous with the above definition. In natural language processing, it involves identifying the most important sentences in the text that contribute to the main idea and piecing them together to form a shorter text.

Abstractive summarization: Another technique that uses natural language to understand the general idea of a text. The text is then rewritten in a different and shorter way while still maintaining the original idea of the full text.

While a variety of LLMs are used to generate custom summaries automatically, one example of a text summarization system is GPT 3.5 developed by OPENAI.

Topic modeling 1018. In statistics and natural language processing, a topic model is a type of statistical model for discovering the abstract “topics” that occur in a collection of documents or texts. Topic modeling is a frequently used text-mining tool for discovery of hidden semantic structures in a text body. Intuitively, given that a document is about a particular topic, one would expect particular words to appear in the document more or less frequently: “dog” and “bone” will appear more often in documents about dogs, “cat” and “meow” will appear in documents about cats, and “the” and “is” will appear approximately equally in both. A document typically concerns multiple topics in different proportions; thus, in a document that is 10% about cats and 90% about dogs, there would probably be about 9 times more dog words than cat words. The “topics” produced by topic modeling techniques are clusters of similar words. A topic model captures this intuition in a mathematical framework, which allows examining a set of documents and discovering, based on the statistics of the words in each, what the topics might be and what each document's balance of topics is.

Topic models are also referred to as probabilistic topic models, which refers to statistical algorithms for discovering the latent semantic structures of an extensive text body. In the age of information, the amount of the written material we encounter each day is simply beyond our processing capacity. Topic models can help to organize and offer insights for us to understand large collections of unstructured text bodies. Originally developed as a text-mining tool, topic models have been used to detect instructive structures in data such as genetic information, images, and networks. They also have applications in other fields such as bioinformatics and computer vision.

Language translation 1020. Finally, algorithms in the AI tool may identify words within an ASR document and which language they belong to. The AI tool can then translate such words into a target language.

The output of the AI tool 1000 can then be fed into the home healthcare, remote patient monitoring, clinician outcome assessment (COA) and clinical workflow automation (CWA) applications. In addition, middleware devices 1050 may be utilized to present the processed ASR text so that it integrates with an electronic health system or EHR 1052 by generating diagnostic reports, scans and prescriptions and medical history of the patient as non-limiting examples.

In general, the AI tool, regardless of source, should aim to leverage AI in healthcare to improve patient outcomes by analyzing healthcare provider-patient and healthcare provider-healthcare prover conversations for admissions, treatment and follow up of patients. For instance, a specific focus may be using a quality of life follow up questionnaire in post-surgical patients as a non-limiting example.

The word “exemplary” is used herein to mean “serving as an example, instance, or illustration.” Any aspect described herein as “exemplary” is not necessarily to be construed as preferred or advantageous over other aspects.

One or more components may reside within a process and/or thread of execution, and a component may be localized on one computer and/or distributed between two or more computers. In addition, these components may execute from various computer readable media having various data structures stored thereon. The components may communicate by way of local and/or remote processes such as in accordance with a signal having one or more data packets (e.g., data from one component interacting with another component in a local system, distributed system, and/or across a network such as the Internet with other systems by way of the signal).

In this description, the terms “telecommunications device,” “communication device,” “wireless device,” “wireless telephone,” “wireless communication device” and “wireless handset” are used interchangeably. With the advent of third generation (“3G”), fourth generation (“4G”), fifth generation (“5G”), and beyond wireless technology, greater bandwidth availability has enabled more portable computing devices with a greater variety of wireless capabilities. Therefore, a telecommunications device (“TD”) may include a cellular telephone, a pager, a PDA, a smartphone, a navigation device, a tablet personal computer (“PC”), a hand-held computer with a wireless connection or link, a watch, a chip embedded within an individual and integrated within the brain, nervous system and muscular system, etc.

In this description, the terms “call” and “communication,” in their noun forms, envision any data transmission routed across a network from one device to another including, but not limited to, a voice transmission, a text message, a video message, a page, a data transmission, etc.

Therefore, although selected aspects have been illustrated and described in detail, it will be understood that various substitutions and alterations may be made therein without departing from the spirit and scope of the present invention, as defined by the following claims.

What is claimed is:

1. A method for integrating the provision of health care services, the method comprising the actions of:
providing access to a communications platform over a network, wherein the communications platform comprises an artificial intelligence (“AI”) tool for processing received information and communications;
providing mobile devices serviced by the communications platform to a plurality of healthcare workers, wherein each of the mobile devices includes a unique identifier;
receiving a health service request at the communications platform, the health service request being sent from an originating mobile device and comprising a unique identifier associated with the originating mobile device;
based on the health service request, the communication platform establishing a communication channel between the originating mobile device and one or more participating devices;
subsequently to establishing the communication channel, sending all communicated information through the AI tool;
the AI tool analyzing the received communicated information to make assessments related to the received communicated information; and
interfacing with an electronic healthcare system to provide the received communicated information and the assessments.

2. The method of claim 1, further comprising the action of creating a text file by performing a speech to text translation on voice information received in the communication information over the communication channel.

3. The method of claim 2, further comprising the action of performing diarization to identify speakers for portions of the text in the text file.

4. The method of claim 1, wherein the AI tool analyzes the received communicated information by creating a summarization.

5. The method of claim 1, wherein the AI tool analyzes the received communicated information by performing a sentiment analysis.

6. The method of claim 1, wherein the AI tool analyzes the received communicated information by performing emotion detection.

7. The method of claim 1, wherein the AI tool analyzes the received communicated information by performing named entity recognition.

8. The method of claim 1, wherein the AI tool analyzes the received communicated information by performing topic modeling.

9. The method of claim 1, wherein the AI tool analyzes the received communicated information by performing language translation.

10. The method of claim 1, the received communication information is stored and stored in a history file.

11. The method of claim 1, wherein the one or more participating devices may include one or more voice enabled internet of things (“IOT”) devices and the communications platform may receive communicated information from the voice enabled IOT devices.

12. The method of claim 1, wherein the communications platform comprises a second line service and further performs the action of provisioning the mobile devices with the second line service and the action of receiving a health service request comprises receiving a request from a mobile device through the second line service.

13. The method of claim 12, wherein the health service request is a doctor-patient conversation and action of the AI tool analyzing the received communicated information to make assessments related to the received communicated information comprises diarizing the conversation.

14. The method of claim 12, wherein receiving the health service request may comprise receiving one of a group of health service request comprising: a doctor-patient conversation, a healthcare provider-healthcare provider conversation, a healthcare provider monitoring a wearable device of a patient, and a hospital worker receiving information from a remote service facility.

15. The method of claim 1, wherein the communication platform establishing a communication channel between the originating mobile device and one or more participating devices further comprises:
identifying a communication protocol for the originating mobile device;
identifying a communication protocol for the one or more participating devices; and
performing any conversion necessary for the participating devices to understand the communication transmission from the originating device and vice versa.

16. The method of claim 1, wherein the action of interfacing with an electronic healthcare system to provide the received communicated information and the assessments further comprises:
receiving a next health service request at the communications platform, the health service request being sent from a next originating mobile device and comprising a unique identifier associated with the next originating mobile device;
based on the health service request, the communication platform establishing a communication channel between the next originating mobile device and one or more participating devices; and
wherein the next health service request is to access previously stored data within the electronic healthcare system such that real-time data sharing is available.

17. A system for integrating the provision of health care services, the system comprising:
a communications platform accessible over a network and including an interface to an electronic healthcare system;
an artificial intelligence tool configured to process received information and communications;
a plurality of mobile devices serviced by the communications platform, wherein each of the mobile devices includes a unique identifier;
the communications platform including executable software programs stored within memory accessible to the communications platform, which when executed cause the communications platform to:
receive a health service request from an originating mobile device and comprising a unique identifier associated with the originating mobile device;
based on the health service request, the communication platform establishing a communication channel between the originating mobile device and one or more participating mobile devices;
subsequently to establishing the communication channel, sending all communicated information through the AI tool for being analyzed to make assessments related to the received communicated information; and
provide the received communicated information and the assessments to the interfacing of the electronic healthcare system.

18. The system of claim 17, further comprising a speech to text translator to convert voice information received in the communication information over the communication channel into a text file.

19. The system of claim 18, further comprising a diarization system to identify speakers for portions of the text in the text file.

20. The system of claim 17, wherein the communications platform comprises a second line service and is further configured to provision the mobile devices with the second line service and receive health service requests through the second line service.
